SEQUENCE LISTING (17 sequences)

SEQ ID NO: 1 - protein, 828 aa, synthetic construct
  REGION 1..828: note = Synthetic construct
DSTSTEVIVHYHRFDSNYANWDLWMWPYQPVNGNGAAYEFSGKDDFGVKADVQVPGDDTQVGLIVRTNDWSQKNTSDDLHIDLTKGHEIWIVQGDPNIYYNLSDAQAAATPKVSNAYLDNEKTVLAKLTNPMTLSDGSSGFTVTDKTTGEQIPVTAATNANSASSSEQTDLVQLTLASAPDVSHTIQVGAAGYEAVNLIPRNVLNLPRYYYSGNDLGNVYSNKATAFRVWAPTASDVQLLLYNSETGPVTKQLEMQKSDNGTWKLKVPGNLKNWYYLYQVTVNGKTQTAVDPYVRAISVNATRGMIVDLEDTNPPGWKEDHQQTPANPVDEVIYEVHVRDFSIDANSGMKNKGKYLAFTEHGTKGPDNVKTGIDSLKELGINAVQLQPIEEFNSIDETQPNMYNWGYDPRNYNVPEGAYATTPEGTARITQLKQLIQSIHKDRIAINMDVVYNHTFATQISDFDKIVPEYYYRTDDAGNYTNGSGTGNEIAAERPMVQKFIIDSLKYWVNEYHIDGFRFDLMALLGKDTMSKAASELHAINPGIALYGEPWTGGTSALPEDQLLTKGAQKGMGVAVFNDNLRNALDGNVFDSSAQGFATGATGLTDAIKNGVEGSINDFTSSPGETINYVTSHDNYTLWDKIALSNPNDSEADRIKMDELAQAVVMTSQGVPFMQGGEEMLRTKGGNDNSYNAGDTVNEFDWSRKAQYPDVFNYYSGLIHLRLDHPAFRMTTANEINSHLQFLNSPENTVAYELTDHVNKDKWGNIIVVYNPNKTAATINLPSGKWAINATSGKVGESTLGQAEGSVQVPGISMMILHQEVSPDHGKK

SEQ ID NO: 2 - DNA, 2586 bp, synthetic construct
  misc_feature 1..2586: note = Synthetic construct
  source 1..2586: mol_type = other DNA
atgtccctaatacgttctaggtataatcattttgtcattctttttactgtcgccataatgtttctaacagtttgtttccccgcttataaagctttagcagattctacctcgacagaagtcattgtgcattatcatcgttttgattctaactatgcaaattgggatctatggatgtggccatatcaaccagttaatggtaatggagcagcatacgagttttctggaaaggatgattttggcgttaaagcagatgttcaagtgcctggggatgatacacaggtaggtctgattgtccgtacaaatgattggagccaaaaaaatacatcagacgatctccatattgatctgacaaaggggcatgaaatatggattgttcagggggatcccaatatttattacaatctgagtgatgcgcaggctgcagcgactccaaaggtttcgaatgcgtatttggataatgaaaaaacagtattggcaaagctaactaatccaatgacattatcagatggatcaagcggctttacggttacagataaaacaacaggggaacaaattccagttaccgctgcaacaaatgcgaactcagcctcctcgtctgagcagacagacttggttcaattgacgttagccagtgcaccggatgtttcccatacaatacaagtaggagcagccggttatgaagcagtcaatctcataccacgaaatgtattaaatttgcctcgttattattacagcggaaatgatttaggtaacgtttattcaaataaggcaacggccttccgtgtatgggctccaactgcttcggatgtccaattacttttatacaatagtgaaacaggacctgtaaccaaacagcttgaaatgcaaaagagtgataacggtacatggaaactgaaggtccctggtaatctgaaaaattggtattatctctatcaggtaacggtgaatgggaagacacaaacagccgttgacccttatgtgcgtgctatttcagtcaatgcaacacgtggtatgatagtcgatttagaagatacgaatcctcctggatggaaagaagatcatcaacagacacctgcgaacccagtggatgaagtaatctacgaagtgcatgtgcgtgatttttcgattgatgctaattcaggcatgaaaaataaagggaaatatcttgcctttacagaacatggcacaaaaggccctgataacgtgaaaacgggtattgatagtttgaaggaattaggaatcaatgctgttcaattacagccgattgaagaatttaacagcattgatgaaacccaaccaaatatgtataactggggctatgacccaagaaactacaacgtccctgaaggagcgtatgcaactacaccagaaggaacggctcgcattacccagttaaagcaactgattcaaagcattcataaagatcggattgctatcaatatggatgtggtctataatcatacctttgccacgcaaatctctgacttcgataaaattgtaccagaatattattaccgtacggatgatgcaggtaattataccaacggatcaggtactggaaatgaaatcgcagccgaaaggccaatggttcaaaaatttattattgattcccttaagtattgggtcaatgagtatcatattgacggcttccgttttgacttaatggcgctgcttggaaaagacacgatgtcgaaagctgcctcggagcttcatgctattaatccaggaattgcactttacggtgagccatggacgggtggaacctctgcactgccagaagatcagcttctgacaaaaggagctcaaaaaggcatgggagtagcggtgtttaatgacaatttacgaaacgcgttggacggcaatgtctttgattcttccgctcaaggttttgcgacaggtgcaacaggcttaactgatgcaattaagaatggcgttgaggggagtattaatgactttacctcttcaccaggtgagacaattaactatgtcacaagtcatgataactacaccctttgggacaaaatagccctaagcaaccctaatgattccgaagcggatcggattaaaatggatgaactcgcacaagcagttgttatgacctcacaaggtgttccattcatgcaaggcggggaagaaatgcttcgtacaaaaggcggcaacgacaatagttataatgcaggcgatacggtcaatgagtttgattggagcaggaaagctcaatatccagatgttttcaactattatagcgggctaatccaccttcgtcttgatcacccagccttccgcatgacgacagctaatgaaatcaatagccacctccaattcctaaatagtccagagaacacagtggcctatgaattaactgatcatgttaataaagacaaatggggaaatatcattgttgtttataacccaaataaaactgcagcaaccattaatttgccgagcgggaaatgggcaatcaatgctacgagcggtaaggtaggagaatccacccttggtcaagcagagggaagtgtccaagtaccaggtatatctatgatgatccttcatcaagaggtaagcccagaccacggtaaaaagtaa

SEQ ID NO: 3 - protein, 825 aa, synthetic construct
  REGION 1..825: note = Synthetic construct
DSTSTEVIVHYHRFDSNYAGWDLWMWPKGGNGAAYEFSGKDDFGVKADVQVPGNPTQVGLIVRTNDWSQKNTSDDLYIDLTKGHEIWIVQGDPNIYYNLSDAQAAATPKVSNAYLDNEKTVLAKLTNPMTLSDGSSGFTVTDKTTGEQIPVTAATNANSASSSEQTDLVQLTLASAPDVSHTIRVGAAGYEGVTLIPRKVLNLPRYYYSGNDLGNVYSPKATAFRVWAPTASDVQLLLYNSETGPVTKALEMRKSDNGTWKLKVPGNLKNWYYLYQVTVNGKTQTAVDPYVRAISVNATRGMIVDLAKTNPPGWKEDHQQTPANPVDEVIYEVHVRDFSIDPNSGMKNKGKYLAFTEHGTKGPDGVKTGIDSLKELGINAVQLLPIEEFASIDETQPNSYNWGYDPRNYNVPEGAYATTPEGTARITEFKQLIQSIHKDRIAINMDVVYNHTWATGVSDFDKIVPEYYYRTDDAGHYTNGSGTGNEIAAERPMVQKFIIDSLKYWVNEYHVDGFRFDLMALLGKDTMSKAASELHAINPGIALYGEPWTGGTSALPRDQLLTKGAQKGMGVAVFNDNLRNALSGNVFDSSAQGFATGATGLTDAIKRGVEGSINDFTSSPSETINYVSCHDNYTLWDKIALSNPNDSEADRIKMDELAQAVVMTSQGVPFMQGGEEMLRTKGGNDNSYNAGDTVNRFDWSRKAQYPDVFNYYSGLIHLRLDHPAFRMTTANEINSHLQFLNSPENTVAYELTDHVNKDKWGNIIVVYNPNKTAATINLPSGKWAINATSGKVGERTLGQAEGSVQVPGISMMILHQEVSPDHGKK

SEQ ID NO: 4 - protein, 828 aa, synthetic construct
  REGION 1..828: note = Synthetic construct
DSTSTEVIVHYHRFDSNYANWDLWMWPYQPVNGNGAAYEFSGKDDFGVKADVQVPGDDTQVGLIVRTNDWSQKNTSDDLHIDLTKGHEIWIVQGDPNIYYNLSDAQAAATPKVSNAYLDNEKTVLAKLTNPMTLSDGSSGFTVTDKTTGEQIPVTAATNANSASSSEQTDLVQLTLASAPDVSHTIQVGAAGYEAVNLIPRNVLNLPRYYYSGNDLGNVYSPKATAFRVWAPTASDVQLLLYNSETGPVTKALEMRKSDNGTWKLKVPGNLKNWYYLYQVTVNGKTQTAVDPYVRAISVNATRGMIVDLEDTNPPGWKEDHQQTPANPVDEVIYEVHVRDFSIDANSGMKNKGKYLAFTEHGTKGPDGVKTGIDSLKELGINAVQLQPIEEFASIDETQPNMYNWGYDPRNYNVPEGAYATTPEGTARITEFKQLIQSIHKDRIAINMDVVYNHTFATQISDFDKIVPEYYYRTDDAGNYTNGSGTGNEIAAERPMVQKFIIDSLKYWVNEYHIDGFRFDLMALLGKDTMSKAASELHAINPGIALYGEPWTGGTSALPEDQLLTKGAQKGMGVAVFNDNLRNALDGNVFDSSAQGFATGATGLTDAIKRGVEGSINDFTSSPSETINYVSCHDNYTLWDKIALSNPNDSEADRIKMDELAQAVVMTSQGVPFMQGGEEMLRTKGGNDNSYNAGDTVNEFDWSRKAQYPDVFNYYSGLIHLRLDHPAFRMTTANEINSHLQFLNSPENTVAYELTDHVNKDKWGNIIVVYNPNKTAATINLPSGKWAINATSGKVGESTLGQAEGSVQVPGISMMILHQEVSPDHGKK

SEQ ID NO: 5 - protein, 828 aa, synthetic construct
  REGION 1..828: note = Synthetic construct
DSTSTEVIVHYHRFDSNYANWDLWMWPYQPVNGNGAAYEFSGKDDFGVKADVQVPGDDTQVGLIVRTNDWSQKNTSDDLYIDLTKGHEIWIVQGDPNIYYNLSDAQAAATPKVSNAYLDNEKTVLAKLTNPMTLSDGSSGFTVTDKTTGEQIPVTAATNANSASSSEQTDLVQLTLASAPDVSHTIRVGAAGYEAVNLIPRNVLNLPRYYYSGNDLGNVYSPKATAFRVWAPTASDVQLLLYNSETGPVTKALEMRKSDNGTWKLKVPGNLKNWYYLYQVTVNGKTQTAVDPYVRAISVNATRGMIVDLAKTNPPGWKEDHQQTPANPVDEVIYEVHVRDFSIDANSGMKNKGKYLAFTEHGTKGPDGVKTGIDSLKELGINAVQLLPIEEFASIDETQPNMYNWGYDPRNYNVPEGAYATTPEGTARITEFKQLIQSIHKDRIAINMDVVYNHTFATGISDFDKIVPEYYYRTDDAGNYTNGSGTGNEIAAERPMVQKFIIDSLKYWVNEYHIDGFRFDLMALLGKDTMSKAASELHAINPGIALYGEPWTGGTSALPEDQLLTKGAQKGMGVAVFNDNLRNALDGNVFDSSAQGFATGATGLTDAIKRGVEGSINDFTSSPSETINYVSCHDNYTLWDKIALSNPNDSEADRIKMDELAQAVVMTSQGVPFMQGGEEMLRTKGGNDNSYNAGDTVNEFDWSRKAQYPDVFNYYSGLIHLRLDHPAFRMTTANEINSHLQFLNSPENTVAYELTDHVNKDKWGNIIVVYNPNKTAATINLPSGKWAINATSGKVGESTLGQAEGSVQVPGISMMILHQEVSPDHGKK

SEQ ID NO: 6 - protein, 484 aa, Bacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRK

SEQ ID NO: 7 - protein, 177 aa, Thermoascus aurantiacus
TRISSCSGSRQSALTTALRNAASLANAAADAAQSGSASKFSEYFKTTSSSTRQTVAARLRAVAREASSSSSGATTYYCDDPYGYCSSNVLAYTLPSYNIIANCDIFYTYLPALTSTCHAQDQATTALHEFTHAPGVYSPGTDDLAYGYQAAMGLSSSQAVMNADTYALYANAIYLGC

SEQ ID NO: 8 - protein, 413 aa, Pyrococcus furiosus
AELEGLDESAAQVMATYVWNLGYDGSGITIGIIDTGIDASHPDLQGKVIGWVDFVNGRSYPYDDHGHGTHVASIAAGTGAASNGKYKGMAPGAKLAGIKVLGADGSGSISTIIKGVEWAVDNKDKYGIKVINLSLGSSQSSDGTDALSQAVNAAWDAGLVVVVAAGNSGPNKYTIGSPAAASKVITVGAVDKYDVITSFSSRGPTADGRLKPEVVAPGNWIIAARASGTSMGQPINDYYTAAPGTSMATPHVAGIAALLLQAHPSWTPDKVKTALIETADIVKPDEIADIAYGAGRVNAYKAINYDNYAKLVFTGYVANKGSQTHQFVISGASFVTATLYWDNANSDLDLYLYDPNGNQVDYSYTAYYDFEKVGYYNPTDGTWTIKVVSYSGSANYQVDVVSDGSLSQPGSSP

SEQ ID NO: 9 - protein, 325 aa, Palaeococcus ferrophilus
GKPKPSQPPQEVPWGIERVKAPSVWSTTDGSSNGVIQVAILDTGIDYDHPDLAANLAWGVSTLRGRVSTKPKDYRDQNGHGTHVAGTIAALNNDIGVVGVAPGVQIYAIRVLDASGRGSYSDIAIGIEQAILGPDGVADKDGDGIIAGDPDDDAAEVISMSLGGSADDSYLHDMIIQAYNAGIVIVAASGNEGASSPSYPAAYPEVIAVGASDINDNIASFSNRQPEVSAPGVDVLSTYPDDTYKTLSGTSMATPHVSGVVALIQAAHFNKYGTILPVGTFDDMSKNTVRGILHITADDLGSPGWDVDYGYGIVRADLAVQAALG

SEQ ID NO: 10 - protein, 598 aa, Talaromyces emersonii
RAPVAARATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 11 - protein, 555 aa, Pycnoporus sanguinea
QSSAVDAYVASESPIAKQGVLNNIGPNGSKAHGAKAGIVVASPSTENPDYLYTWTRDSSLVFKLLIDQFTSGDDTSLRGLIDDFTSAEAILQQVSNPSGTVSTGGLGEPKFNIDETAFTGAWGRPQRDGPALRATSIIRYANWLLDNGNTTYVSNTLWPVIQLDLDYVADNWNQSTFDLWEEVDSSSFFTTAVQHRALREGATFASRIGQSSVVSGYTTQADNLLCFLQSYWNPSGGYVTANTGGGRSGKDSNTVLTSIHTFDPAAGCDAATFQPCSDKALSNLKVYVDAFRSIYTINNGIASNAAVATGRYPEDSYMGGNPWYLTTSAVAEQLYDALYVWDQLGGLNVTSTSLAFFQQFASGLSTGTYSASSSTYATLTSAIRSFADGFLAINAKYTPADGGLAEQYSRNDGTPLSAVDLTWSYAAALTAFAAREGKTYGSWGAAGLTVPASCSGGGGATVAVTFNVQATTVFGENIYITGSVAALQNWSPDNALILSAANYPTWSITVNLPANTVVQYKYIRKFNGQVTWESDPNNQITTPSGGSFTQNDVWR

SEQ ID NO: 12 - protein, 556 aa, Gloeophyllum sepiarium
QSVDSYVSSEGPIAKAGVLANIGPNGSKASGASAGVVVASPSTSDPDYWYTWTRDSSLVFKSLIDQYTTGIDSTSSLRTLIDDFVTAEANLQQVSNPSGTLTTGGLGEPKFNVDETAFTGAWGRPQRDGPALRSTALITYGNWLLSNGNTSYVTSNLWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYVDSFRSVYSINSGVASNAAVATGRYPEDSYQGGNPWYLTTFAVAEQLYDALNVWESQGSLEVTSTSLAFFQQFSSGVTAGTYSSSSSTYSTLTSAIKNFADGFVAINAKYTPSNGGLAEQYSKSDGSPLSAVDLTWSYASALTAFEARNNTQFAGWGAAGLTVPSSCSGNSGGPTVAVTFNVNAETVWGENIYLTGSVDALENWSADNALLLSSANYPTWSITVNLPASTAIEYKYIRKNNGAVTWESDPNNSITTPASGSTTENDTWR

SEQ ID NO: 13 - protein, 559 aa, Gloeophyllum trabeum
QSVDSYVGSEGPIAKAGVLANIGPNGSKASGAAAGVVVASPSKSDPDYWYTWTRDSSLVFKSLIDQYTTGIDSTSSLRSLIDSFVIAEANIQQVSNPSGTLTTGGLGEPKFNVDETAFTGAWGRPQRDGPALRATALITYGNWLLSNGNTTWVTSTLWPIIQNDLNYVVQYWNQTTFDLWEEVNSSSFFTTAVQHRALREGAAFATKIGQTSSVSSYTTQAANLLCFLQSYWNPTSGYITANTGGGRSGKDANTLLASIHTYDPSAGCDATTFQPCSDKALSNLKVYVDSFRSVYSINSGIASNAAVATGRYPEDSYQGGNPWYLTTFAVAEQLYDALNVWAAQGSLNVTSISLPFFQQFSSSVTAGTYASSSTTYTTLTSAIKSFADGFVAINAQYTPSNGGLAEQFSRSNGAPVSAVDLTWSYASALTAFEARNNTQFAGWGAVGLTVPTSCSSNSGGGGGSTVAVTFNVNAQTVWGENIYITGSVDALSNWSPDNALLLSSANYPTWSITVNLPASTAIQYKYIRKNNGAVTWESDPNNSITTPASGSVTENDTWR

SEQ ID NO: 14 - protein, 556 aa, Trametes cingulata
QSSAADAYVASESPIAKAGVLANIGPSGSKSNGAKAGIVIASPSTSNPNYLYTWTRDSSLVFKALIDQFTTGEDTSLRTLIDEFTSAEAILQQVPNPSGTVSTGGLGEPKFNIDETAFTDAWGRPQRDGPALRATAIITYANWLLDNKNTTYVTNTLWPIIKLDLDYVASNWNQSTFDLWEEINSSSFFTTAVQHRALREGATFANRIGQTSVVSGYTTQANNLLCFLQSYWNPTGGYITANTGGGRSGKDANTVLTSIHTFDPAAGCDAVTFQPCSDKALSNLKVYVDAFRSIYSINSGIASNAAVATGRYPEDSYMGGNPWYLTTSAVAEQLYDALIVWNKLGALNVTSTSLPFFQQFSSGVTVGTYASSSSTFKTLTSAIKTFADGFLAVNAKYTPSNGGLAEQYSRSNGSPVSAVDLTWSYAAALTSFAARSGKTYASWGAAGLTVPTTCSGSGGAGTVAVTFNVQATTVFGENIYITGSVPALQNWSPDNALILSAANYPTWSITVNLPASTTIEYKYIRKFNGAVTWESDPNNSITTPASGTFTQNDTWR

SEQ ID NO: 15 - protein, 583 aa, synthetic construct
  REGION 1..583: note = Synthetic construct
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 16 - protein, 595 aa, Penicillium oxalicum
RPDPKGGNLTPFIHKEGERSLQGILDNLGGRGKKTPGTAAGLFIASPNTENPNYYYTWTRDSALTAKCLIDLFEDSRAKFPIDRKYLETGIRDYKSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 17 - protein, 674 aa, Myceliophthora sepedonium
LYINGSVTAPCDSPIYCQGELLKAVELARPFVDSKTFVDMPTIKPVDEVLAAFSKLSLPLSNNSELNAFLYENFAQAGHELEEVPDSELETDAKFLDKLEDRTIKEFVGKVIDIWPDLTRRYAGPSNCTECANSFIPVNRTFVVAGGRFREPYYWDSYWIVEGLLRTGGAFTHISKNIIENFLDFVDTIGFIPNGARIYYLNRSQPPLLTLMVKSYVDYTNDTSILDRALPLLIKEHEFFMNNRTVSITGSNGKEYTLNRYHVENNQPRPESFREDYITANNGSYYASSGIIYPVKTPLNETEKAALYSNLATGAESGWDYTSRWLGVPSDAARDVYFPLRSLNVRDIVPVDLNSILYQNEVIIAEYLEKAGNSSAAKRFATAAEQRSEAMYSLMWNATHWSYFDYNLTDNTQHIFVPADEDTAPQDRIEAPPGQQVFFHIAQLYPFWTGAAPASLKANPLAVQQAYARVARMLDIKKGAIPATNYRTGQQWDQPNVWPPLQHILMKGLLNTPATFGKSDPAYQSVQNLALRLAQRYLDSTFCTWYATGGSTSDFPQLEGVTPGATGVMFEKYADNATNVAGGGGEYEVVEGFGWTNGVLIWAADVFGNKLKRPDCGNITAAHTHSSAKRGLEENKLPRRAVELDPWDAAWTKMFGRSKLRRREAEDVRKRWMS

(19) United States
(12) Patent Application Publication
(10) Pub. No.: US 2025/0305006 A1
(43) Pub. Date: Oct. 2, 2025

(54) Polypeptides Having Pullulanase Activity Suitable For Use In Liquefaction

(71) Applicant: Novozymes A/S, Bagsvaerd (DK)

(72) Inventors: Takashi Nakanishi, Chiba-shi (JP); Aki Tomiki, Chiba-shi (JP); Yuma Kurakata, Chiba-shi (JP)

(73) Assignee: Novozymes A/S, Bagsvaerd (DK)

(21) Appl. No.: 19/235,254

(22) Filed: Jun. 11, 2025

Related U.S. Application Data
(62) Division of application No. 17/440,477, filed on Sep. 17, 2021, now Pat. No. 12,359,227.

(30) Foreign Application Priority Data
Mar. 18, 2019 (EP) 19163366.8

(51) Int. Cl. C12P 7/06; C12N 9/44

(52) U.S. Cl. CPC C12P 7/06; C12N 9/2457; C12Y 302/01041

(57) ABSTRACT

The present invention relates to a variant pullulanase, having increased thermo-stability and/or increased thermo-activity compared to a parent pullulanase, comprising a substitution at least a one position selected from a position corresponding to positions 432, 486, 370, 17, 77, 103, 106, 107, 190, 196, 197, 262, 279, 283, 321, 367, 375, 382, 399, 401, 402, 411, 412, 434, 435, 443, 446, 459, 460, 479, 490, 498, 514, 529, 531, 533, 541, 545, 581, 583, 595, 649, 665, 688, 700, 709, 804, 811 of SEQ ID NO: 1, and optionally a deletion of one or more, e.g., all amino acids at positions 821, 822, 823, 824, 825, 826, 827, and 828, wherein the variant has pullulanase activity, and wherein the variant has at least 70%, at least 75%, at least 80%, at least 85%, at least 90%, at least 95%, at least 96%, at least 97%, at least 98%, or at least 99%, but less than 100% sequence identity to a parent alpha amylase selected from the group consisting of SEQ ID NO: 1, SEQ ID NO: 3, SEQ ID NO: 4, SEQ ID NO: 5.

Further aspect the present invention relates to a process for liquefying starch-containing material at a temperature above the initial gelatinization temperature using an alpha-amylase and a thermo-stable pullulanase of the invention.

### REFERENCE TO A SEQUENCE LISTING

This application contains a Sequence Listing in computer readable form, which is incorporated herein by reference.

### FIELD OF THE INVENTION

The present invention relates to use of thermo-stable pullulanase variants in a process for producing fermentation products from starch-containing material and to variant polypeptides having pullulanase activity.

### BACKGROUND OF THE INVENTION

Starch usually consists of about 80% amylopectin and 20% amylose. Amylopectin is a branched polysaccharide in which linear chains alpha-1,4 D-glucose residues are joined by alpha-1,6 glucosidic linkages. Amylopectin is partially degraded by alpha-amylase, which hydrolyzes the 1,4-alpha-glucosidic linkages to produce branched and linear oligosaccharides. Prolonged degradation of amylopectin by alpha-amylase results in the formation of so-called alpha-limit dextrins that are not susceptible to further hydrolysis by the alpha-amylase. Branched oligosaccharides can be hydrolyzed into linear oligosaccharides by a debranching enzyme. The remaining branched oligosaccharides can be depolymerized to D-glucose by glucoamylase, which hydrolyzes linear oligosaccharides into D-glucose.

Debranching enzymes which can attack amylopectin are divided into two classes: isoamylases (E.C. 3.2.1.68) and pullulanases (E.C. 3.2.1.41), respectively. Isoamylase hydrolyses alpha-1,6-D-glucosidic branch linkages in amylopectin and beta-limit dextrins and can be distinguished from pullulanases by the inability of isoamylase to attack pullulan, and by their limited action on alpha-limit dextrins.

It is well-known in the art to add isoamylases or pullulanases in starch conversion processes. Pullulanase is a starch debranching enzyme having pullulan 6-glucano-hydrolase activity (EC3.2.1.41) that catalyzes the hydrolyses the α-1,6-glycosidic bonds in pullulan, releasing maltotriose with reducing carbohydrate ends. Usually pullulanase is used in combination with an alpha amylase and/or a glucoamylase.

Pullulanases are known in the art. U.S. Pat. Nos. 6,074,854 and 5,817,498 disclose a pullulanase from Bacillus deramificans. WO2009/075682 discloses a pullulanase derived from Bacillus acidopullulyticus.

WO 2015/007639 discloses a hybrid pullulanase obtained by combining an N-terminal fragment of a pullulanase from Bacillus acidopullulyticus fused to a C-terminal fragment of a pullulanase from Bacillus deramificans. Prior art pullulanases derived from Bacillus sp. have so far not been sufficiently thermo-stable for use in liquefaction in conventional starch conversion processes.

WO2015/110473 and WO2017/014974 disclose thermo-stabilized pullulanase variants.

It is an object of the present invention to provide pullulanase variants having increased thermo-stability and/or thermo-activity suitable for use in liquefaction of starch containing material.

### SUMMARY OF THE INVENTION

The present invention relates to a variant pullulanase, having increased thermo-stability and/or increased thermo-activity compared to a parent pullulanase, comprising a substitution at least at one position selected from a position corresponding to positions 432, 486, 370, 17, 77, 103, 106, 107, 190, 196, 197, 262, 279, 283, 321, 367, 375, 382, 399, 401, 402, 411, 412, 434, 435, 443, 446, 459, 460, 479, 490, 498, 514, 529, 531, 533, 541, 545, 581, 583, 595, 649, 665, 688, 700, 709, 804, 811 of SEQ ID NO: 1, and optionally a deletion of one or more, e.g., all amino acids at positions 821, 822, 823, 824, 825, 826, 827, and 828, wherein the variant has pullulanase activity, and wherein the variant has at least 70%, at least 75%, at least 80%, at least 85%, at least 90%, at least 95%, at least 96%, at least 97%, at least 98%, or at least 99%, but less than 100% sequence identity to a parent alpha amylase selected from the group consisting of SEQ ID NO: 1, SEQ ID NO: 3, SEQ ID NO: 4, SEQ ID NO: 5.

Further aspect the present invention relates to a process for liquefying starch-containing material at a temperature above the initial gelatinization temperature using an alpha-amylase and a thermo-stable pullulanase of the invention.

Thus, in a second aspect the invention relates to a process for producing a syrup from starch-containing material comprising the steps of:
a) liquefying the starch-containing material at a temperature above the initial gelatinization temperature using an alpha-amylase and a variant pullulanase of the invention;
b) saccharifying using a glucoamylase.

In a third aspect the present invention relates to a process for producing fermentation products from starch-containing material comprising the steps of:
a) liquefying the starch-containing material at a temperature above the initial gelatinization temperature using an alpha-amylase and a variant pullulanase of the invention;
b) saccharifying using a glucoamylase;
c) fermenting using a fermenting organism.

In a fourth aspect the present invention relates to compositions comprising the variant pullulanase of the invention and a stabilizer.

The present invention also relates to polynucleotides encoding the variants; nucleic acid constructs, vectors, and host cells comprising the polynucleotides; and methods of producing the variants.

Furthermore, the present invention relates to use of the variant pullulanase of the invention in liquefaction of starch-containing material.

### Definitions

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to one or more control sequences that provide for its expression. Each control sequence may be native (i.e., from the same gene) or foreign (i.e., from a different gene) to the polynucleotide encoding the polypeptide

Fragment: The term “fragment” means a polypeptide having one or more (e.g., several) amino acids absent from the amino and/or carboxyl terminus of a mature polypeptide or domain; wherein the fragment has pullulanas activity.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance). An isolated substance may be present in a fermentation broth sample; e.g. a host cell may be genetically modified to express the polypeptide of the invention. The fermentation broth from that host cell will comprise the isolated polypeptide.

Mature polypeptide: The term “mature polypeptide” means a polypeptide in its final form following translation and any post-translational modifications, such as N-terminal processing and C-terminal truncation.

Mature polypeptide coding sequence: The term “mature polypeptide coding sequence” means a polynucleotide that encodes a mature polypeptide having pullulanase activity.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Pullulanase: The term “pullulanase” means a starch debranching enzyme having pullulan 6-glucano-hydrolase activity (EC 3.2.1.41) that catalyzes the hydrolysis the α-1,6-glycosidic bonds in pullulan, releasing maltotriose with reducing carbohydrate ends. For purposes of the present invention, pullulanase activity can be determined according to the procedure described in the Examples. In the context of the present invention the variant pullulanases have increased thermo-activity and or increased thermo-stability. Pullulanase activity was determined (using the PHADEBAS assay) as relative activity after heat stress/shock for 30 min at two different temperatures in the range from 60-90° C., e.g., 70-87° C., and assayed at a temperature in the range from 60° C.-80° C., e.g., 70° C., depending on the thermo-stability of the variant (thermo-stability), or as relative activity determined at two different temperatures (70-86° C.) (thermoprofile/thermo-activity) as described in the examples. Increased thermo-stability was also measured using the TSA assay for determining melting/denaturing temperature of the variant polypeptides.

Wild-type Pullulanase: The term “wild-type” pullulanase means a pullulanase expressed by a naturally occurring microorganism, such as a bacterium, yeast, or filamentous fungus found in nature.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

The term “low stringency conditions” means for probes of at least 100 nucleotides in length. prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

Subsequence: The term “subsequence” means a polynucleotide having one or more (e.g., several) nucleotides absent from the 5′ and/or 3′ end of a mature polypeptide coding sequence; wherein the subsequence encodes a fragment having pullulanase activity.

S8A Protease: The term “S8A protease” means an S8 protease belonging to subfamily A. Subtilisins, EC 3.4.21.62, are a subgroup in subfamily S8A.

Variant: The term “variant” means a polypeptide having pullulanase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. In describing variants, the nomenclature described below is adapted for case of reference. The accepted IUPAC single letter or three letter amino acid abbreviations are employed.

In the context of the present invention the variant pullulanases has increased thermo-stability and/or increased thermo-activity.

Thermo-stability may be determined (using the PHADEBAS assay) as relative activity after heat stress/shock for 30 min at two different temperatures in the range from 60-90° C., e.g., 70-87° C., and assayed at a temperature in the range from 60° C.-80° C., e.g., 70° C., depending on the thermo-stability of the variant (thermo-stability), or as relative activity determined at two different temperatures (70-86° C.) (thermoprofile/thermo-activity) as described in the examples. Increased thermo-stability may also be measured using the TSA assay for determining melting/denaturing temperature of the variant polypeptides. In one embodiment the pullulanase variants of the invention have an increase in thermo-stability determined as increased melting (denaturing) temperature compared to the parent pullulanase disclosed in SEQ ID NO: 3 using TSA assay of at least 0.3 degrees C., at least 0.4 degrees C., at least 0.5 degrees C., at least 0.6 degrees C., at least 0.8 degrees C., at least 1.0 degrees C., at least 1.2 degrees C., at least 1.5 degrees C., at least 2.0 degrees C., at least 2.5 degrees C., at least 3.0 degrees C., at least 3.5 degrees C., at least 4.0 degrees C., at least 4.5 degrees C., at least 5.0 degrees C.

Increased thermo-stability was measured as described in the examples using the PHADEBAS assay by heat-shock for e.g., 30 min at a temperature in the range from 70-87° C. and then activity was assayed at e.g., 70° C. or 80° C. Thermo-stability was then determined as relative activity of the sample heat-shocked at the higher temperature over the activity of the sample heat-shocked at the lower temperature. E.g., for variant P609 (table 1a of example 2) when heat-shocked at 81.5° C. and at 80° C. the relative activity was 58%, meaning that after incubation at 81.5° C. the activity was 58% compared to the sample incubated at 80° C. Activity was then calculated as relative activity to the parent pullulanase, JPUL604 (SEQ ID NO: 3). The skilled person will know what will be an appropriate temperature to use for heat-shock/stress and for activity assay since this will depend on the thermo-stability of the parent pullulanase and of the resulting variant.

Increased thermo-activity (thermo-profile) was determined as relative activity using the PHADEBAS assay by performing the activity assay at two different temperatures, e.g., in the range 70-86° C., and calculating the % activity at the higher temperature compared to the lower temperature. In some examples thermo-activity was determined by enzymatic reaction with the substrate maltodextrin/pullulan (DE3) at high temperature e.g. 2 hours at 85° C. or 30 min 91° C. Subsequently, the pullulanase digested fraction of maltodextrin was measured by PAHBAH assay at 55° C.

In one embodiment pullulanase variants of the invention have an increase in thermo-activity relative to a parent pullulanase, e.g., the pullulanase disclosed as SEQ ID NO: 3, of at least 5%, at least 10%, at least 20%, at least 25%, at least 30%, at least 35%, at least 40%, at least 50%, at least 60%, at least 70%, at least 80%, at least 90%, at least 100%, after enzymatic reaction of maltodextrin 2 hours at 85° C. or 30 min 91° C., and subsequent determination of digested maltodextrin fraction by PAHBAH assay at 55° C.

### Conventions for Designation of Variants

For purposes of the present invention, the mature hybrid pullulanase polypeptide disclosed as SEQ ID NO: 1 is used to determine the corresponding amino acid residue in another pullulanase. The amino acid sequence of another pullulanase is aligned with the mature polypeptide disclosed as SEQ ID NO: 1, and based on the alignment, the amino acid position number corresponding to any amino acid residue in the mature polypeptide disclosed as SEQ ID NO: 1 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another pullulanase can be determined by an alignment of multiple polypeptide sequences using several computer programs including, but not limited to, MUSCLE (multiple sequence comparison by log-expectation: version 3.5 or later: Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other enzyme has diverged from the polypeptide of SEQ ID NO: 1 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for case of reference. The accepted IUPAC single letter or three letter amino acid abbreviations are employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions. For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a |
| | 195b |
| G | G - K - A |

Multiple alterations. Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different alterations. Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants:

“Tyr167Gly+Arg170Gly”, “Tyr167Gly+Arg170Ala”, “Tyr167Ala+Arg170Gly”, and “Tyr167Ala+Arg170Ala”.

Throughout the present description in some embodiments the variants of the invention have been described by giving the amino acid present at the specified position in SEQ ID NO: 1 as well as the amino acid present after substitution. This does not mean that the starting amino acid in the specified position cannot be a different one. The starting amino acid in a specific position of course depends on the choice of the parent pullulanase, thus in the present disclosure sometimes the starting amino acid has been denoted X, meaning that this could be any amino acid. The essential feature of the present invention is the resulting amino acid present after the substitution.

### DETAILED DESCRIPTION OF THE INVENTION

The present invention relates to variant pullulanases derived from a hybrid parent pullulanase. The hybrid pullulanase, disclosed as SEQ ID NO: 1 herein, was used as the parent pullulanase. The polynucleotide sequence encoding the parent pullulanase is included herein as SEQ ID NO: 2, wherein nucleotides 1-99 encode a signal peptide, and nucleotides 100-2583 encode the mature polypeptide disclosed in SEQ ID NO: 1. In other embodiments the parent pullulanase is selected from the pullulanases disclosed in SEQ ID NO: 3, SEQ ID NO: 4, and SEQ ID NO: 5.

The variants according to the present invention have improved properties compared to the parent. The improved properties are selected from increased thermo-activity (thermo-profile), and/or increased thermo-stability. Pullulanase activity may be determined using any suitable pullulanase assay, such as e.g., by the PHADEBAS assay, or the PAHBAH-pullulan assay described herein in the pullulanase assay section and examples.

Particularly, the present invention relates to a pullulanase variant, having increased thermo-stability and/or increased thermo-activity compared to a parent pullulanase, comprising a substitution at least at one position selected from a position corresponding to positions 432, 486, 370, 17, 77, 103, 106, 107, 190, 196, 197, 262, 279, 283, 321, 367, 375, 382, 399, 401, 402, 411, 412, 434, 435, 443, 446, 459, 460, 479, 490, 498, 514, 529, 531, 533, 541, 545, 581, 583, 595, 649, 665, 688, 700, 709, 804, 811 of SEQ ID NO: 1, and optionally a deletion of one or more, e.g., all amino acids at positions 821, 822, 823, 824, 825, 826, 827, and 828, wherein the variant has pullulanase activity, and wherein the variant has at least 70%, at least 75%, at least 80%, at least 85%, at least 90%, at least 95%, at least 96%, at least 97%, at least 98%, or at least 99%, but less than 100% sequence identity to a parent alpha amylase selected from the group consisting of SEQ ID NO: 1, SEQ ID NO: 3, SEQ ID NO: 4, SEQ ID NO: 5.

Particularly the substitutions are selected from the group consisting of: K370S, F17Y, D77G, S103K, Q106W, A107D, A190I, V196T,C. T197I, T262V, Q279R, N283F, H321V, D367G,N, S375H, N382T, Q399N, N401D, S402Q, N411L, Y412F, F432V, Q434E, L435A, R443G, I446V, G459E, V460E, H479N, T486A,V, I490L, Q498R, V514A, T529L, S531R, A533I, N541D, A545I, L581F, N583D, Q595R, D649A, V665I, D688A, F700L, P709I, E804S, and G811R.

In one aspect the present invention therefore relates to a pullulanase variant, having increased thermo-stability and/or increased thermo-activity compared to a parent pullulanase, comprising a substitution at a position corresponding to position 370 of SEQ ID NO: 1, wherein the variant pullulanase comprises a serine in position 370 using SEQ ID NO: 1 for numbering, particularly a K370S substitution, wherein the variant has pullulanase activity, and wherein the variant has at least 70%, at least 75%, at least 80%, at least 85%, at least 90%, at least 95%, at least 96%, at least 97%, at least 98%, or at least 99%, but less than 100% sequence identity to a parent alpha amylase selected from the group consisting of SEQ ID NO: 1, SEQ ID NO: 3, SEQ ID NO: 4, SEQ ID NO: 5.

In another aspect the present invention therefore relates to a pullulanase variant, having increased thermo-stability and/or increased thermo-activity compared to a parent pullulanase, comprising a substitution at a position corresponding to position 432 of SEQ ID NO: 1, wherein the variant pullulanase comprises a valine in position 432 using SEQ ID NO: 1 for numbering, particularly a F432V substitution, wherein the variant has pullulanase activity, and wherein the variant has at least 70%, at least 75%, at least 80%, at least 85%, at least 90%, at least 95%, at least 96%, at least 97%, at least 98%, or at least 99%, but less than 100% sequence identity to a parent alpha amylase selected from the group consisting of SEQ ID NO: 1, SEQ ID NO: 3, SEQ ID NO: 4, SEQ ID NO: 5.

In another aspect the present invention therefore relates to a pullulanase variant, having increased thermo-stability and/or increased thermo-activity compared to a parent pullulanase, comprising a substitution at a position corresponding to position 486 of SEQ ID NO: 1, wherein the variant pullulanase comprises an alanine or a valine in position 486 using SEQ ID NO: 1 for numbering, particularly a T486A,V substitution, wherein the variant has pullulanase activity, and wherein the variant has at least 70%, at least 75%, at least 80%, at least 85%, at least 90%, at least 95%, at least 96%, at least 97%, at least 98%, or at least 99%, but less than 100% sequence identity to a parent alpha amylase selected from the group consisting of SEQ ID NO: 1, SEQ ID NO: 3, SEQ ID NO: 4, SEQ ID NO: 5.

The increase in thermo-stability may in one embodiment be determined as relative activity after heat stress for 30 min at two different temperatures selected in the range from 60° C.-90° C., e.g., 70° C.-87° C., and subsequently assayed at 60-80° C., such as 70° C. using PHADEBAS assay. Alternatively, thermo-stability may be determined as increased melting (denaturing) temperature compared to the parent pullulanase using TSA assay.

The increase in thermo-activity (thermo profile) may in one embodiment be determined as relative activity determined at two different temperatures selected from the range of 70-86° C. using PHADEBAS assay.

In a further specific embodiment the variant pullulanase of the invention relates to a pullulanase variant, having increased thermo-stability and/or increased thermo-activity compared to a parent pullulanase, comprising a substitution at least at one position selected from a position corresponding to positions 432, 486, 370, 17, 77, 103, 106, 107, 190, 196, 197, 262, 279, 283, 321, 367, 375, 382, 399, 401, 402, 411, 412, 434, 435, 443, 446, 459, 460, 479, 490, 498, 514, 529, 531, 533, 541, 545, 581, 583, 595, 649, 665, 688, 700, 709, 804, 811 of SEQ ID NO: 1, and optionally a deletion of one or more, e.g., all amino acids at positions 821, 822, 823, 824, 825, 826, 827, and 828, wherein the variant has pullulanase activity, and wherein the variant has at least 70%, at least 75%, at least 80%, at least 85%, at least 90%, at least 95%, at least 96%, at least 97%, at least 98%, or at least 99%, but less than 100% sequence identity to a parent alpha amylase selected from the group consisting of SEQ ID NO: 1, SEQ ID NO: 3, SEQ ID NO: 4, SEQ ID NO: 5, and wherein the variant comprises combinations of substitutions and/or deletions selected from the group consisting of:
Q279R+K370S;
H321E+K370S;
K370S+V821*+S822*+P823*+D824*+H825*+G826*+K827*+K828*;
K370S+Q399N;
K370S+N401D;
K370S+F432V;
V196T+K370S;
V196C+K370S;
T197I+K370S;
K370S+V460E;
K370S+T486A;
K370S+T486V;
K370S+I490L;
K370S+V514A;
K370S+T529L;
K370S+S531R;
K370S+Q595R;
H321E+K370S+F432V;
K370S+F432V+V821*+S822*+P823*+D824*+H825*+G826*+K827*+K828*;
K370S+Q399N+F432V;
K370S+N401D+F432V;
V196T+K370S+F432V;
V196C+K370S+F432V;
T197I+K370S+F432V;
K370S+F432V+G459E;
K370S+F432V+T486A;
K370S+F432V+T486V;
V196T+K370S+F432V+T486A;
T197I+K370S+F432V+T486A;
V196T+T197I+K370S+F432V+T486A;
K370S+Q399N+F432V+T486A;
K370S+N401D+F432V+T486A;
K370S+Q399N+N401D+F432V+T486A;
K370S+S531R+F432V+T486A;
K370S+Q595R+F432V+T486A;
K370S+F432V+T486A+S531R+Q595R;
V196T+T197I+K370S+Q399N+N401D+F432V+T486A;
V196T+T197I+K370S+Q399N+N401D+F432V+T486A+Q595R;
T197I+K370S+N401D+F432V+T486A+S531R+Q595R;
T197I+K370S+N401D+F432V+T486A+S531R+Q595R+V821*+S822*+P823*+D824*+H825*+G826*+K827*+K828*;
T197I+K370S+Q399N+N401D+F432V+T486A+Q595R;
S103K+T197I+K370S+N401D+F432V+T486A+S531R+Q595R;
V196T+T197I+K370S+Q399N+N401D+F432V+T486V+Q595R;
T197I+K370S+N401D+F432V+T486V+S531R+Q595R;
V196T+T197I+K370S+Q399N+N401D+F432V+T486A+S531R+Q595R;
D77G+T197I+K370S+N401D+F432V+T486V+S531R+Q595R;
T197I+K370S+N401D+S402Q+F432V+T486V+S531R+Q595R;
T197I+N283F+K370S+N401D+F432V+T486V+S531R+Q595R;
T197I+N283F+K370S+N401D+S402Q+F432V+T486V+S531R+Q595R;
T197I+N283F+K370S+N401D+F432V+L435A+T486V+S531R+Q595R;
D77G+T197I+N283F+K370S+N401D+S402Q+F432V+T486V+S531R+Q595R;
T197I+N283F+K370S+N401D+S402Q+F432V+L435A+T486V+S531R+Q595R;
D77G+T197I+N283F+K370S+N401D+S402Q+F432V+L435A+T486V+S531R+Q595R;
T197I+N283F+K370S+N401D+S402Q+F432V+L435A+T486V+S531R+Q595R+D649A;
D77G+T197I+N283F+K370S+N401D+S402Q+F432V+L435A+T486V+S531R+Q595R+D649A;
D77G+Q106W+T197I+N283F+K370S+N401D+S402Q+F432V+L435A+T486V+S531R+Q595R;
D77G+A107D+T197I+N283F+K370S+N401D+S402Q+F432V+L435A+T486V+S531R+Q595R;
D77G+V196C+T197I+N283F+K370S+N401D+S402Q+F432V+L435A+T486V+S531R+Q595R;
D77G+T197I+N283F+K370S+N401D+S402Q+F432V+L435A+T486V+S531R+N583D+Q595R;
D77G+T197I+N283F+K370S+N401D+S402Q+F432V+L435A+T486V+S531R+Q595R+V821*+S822*+P823*+D824*+H825*+G826*+K827*+K828*;
D77G+T197I+N283F+K370S+N401D+S402Q+F432V+L435A+T486V+S531R+Q595R+D688A+V821*+S822*+P823*+D824*+H825*+G826*+K827*+K828*;
D77G+T197I+N283F+D367G+K370S+N401D+S402Q+F432V+L435A+T486V+S531R+Q595R+V821*+S822*+P823*+D824*+H825*+G826*+K827*+K828*;
D77G+T197I+N283F+D367N+K370S+N401D+S402Q+F432V+L435A+T486V+S531R+Q595R+V821*+S822*+P823*+D824*+H825*+G826*+K827*+K828*;
D77G+T197I+N283F+K370S+S375H+N401D+S402Q+F432V+L435A+T486V+S531R+Q595R+V821*+S822*+P823*+D824*+H825*+G826*+K827*+K828*;
D77G+T197I+N283F+K370S+N382T+N401D+S402Q+F432V+L435A+T486V+S531R+Q595R+V821*+S822*+P823*+D824*+H825*+G826*+K827*+K828*;
D77G+T197I+N283F+K370S+N401D+S402Q+N411L+F432V+L435A+T486V+S531R+Q595R+V821*+S822*+P823*+D824*+H825*+G826*+K827*+K828*;
D77G+T197I+N283F+K370S+N401D+S402Q+Y412F+F432V+L435A+T486V+S531R+Q595R+V821*+S822*+P823*+D824*+H825*+G826*+K827*+K828*;
D77G+T197I+N283F+K370S+N401D+S402Q+F432V+Q434E+L435A+T486V+S531R+Q595R+V821*+S822*+P823*+D824*+H825*+G826*+K827*+K828*;
D77G+T197I+N283F+K370S+N401D+S402Q+F432V+L435A+R443G+T486V+S531R+Q595R+V821*+S822*+P823*+D824*+H825*+G826*+K827*+K828*;
D77G+T197I+N283F+K370S+N401D+S402Q+F432V+L435A+I446V+T486V+S531R+Q595R+V821*+S822*+P823*+D824*+H825*+G826*+K827*+K828*;
D77G+T197I+N283F+K370S+N401D+S402Q+F432V+L435A+T486V+Q498R+S531R+Q595R+V821*+S822*+P823*+D824*+H825*+G826*+K827*+K828*;
D77G+T197I+N283F+K370S+N401D+S402Q+F432V+L435A+T486V+S531R+A533I+Q595R+V821*+S822*+P823*+D824*+H825*+G826*+K827*+K828*;
D77G+T197I+N283F+K370S+N401D+S402Q+F432V+L435A+T486V+S531R+N541D+Q595R+V821*+S822*+P823*+D824*+H825*+G826*+K827*+K828*;
D77G+T197I+N283F+K370S+N401D+S402Q+F432V+L435A+T486V+S531R+A545I+Q595R+V821*+S822*+P823*+D824*+H825*+G826*+K827*+K828*;
D77G+T197I+N283F+K370S+N401D+S402Q+F432V+L435A+T486V+S531R+L581F+Q595R+V821*+S822*+P823*+D824*+H825*+G826*+K827*+K828*;
D77G+T197I+N283F+K370S+N401D+S402Q+F432V+L435A+H479N+T486V+S531R+Q595R+D688A+V821*+S822*+P823*+D824*+H825*+G826*+K827*+K828*;
D77G+T197I+N283F+D367N+K370S+N401D+S402Q+F432V+L435A+T486V+S531R+Q595R+D688A+V821*+S822*+P823*+D824*+H825*+G826*+K827*+K828*;
D77G+T197I+N283F+K370S+S375H+N401D+S402Q+F432V+L435A+T486V+S531R+Q595R+D688A+V821*+S822*+P823*+D824*+H825*+G826*+K827*+K828*;
D77G+T197I+N283F+K370S+N401D+S402Q+Y412F+F432V+L435A+T486V+S531R+Q595R+D688A+V821*+S822*+P823*+D824*+H825*+G826*+K827*+K828*;
D77G+T197I+N283F+K370S+N401D+S402Q+F432V+L435A+T486V+S531R+A533I+Q595R+D688A+V821*+S822*+P823*+D824*+H825*+G826*+K827*+K828*;
D77G+T197I+N283F+K370S+N401D+S402Q+F432V+L435A+T486V+S531R+L581F+Q595R+D688A+V821*+S822*+P823*+D824*+H825*+G826*+K827*+K828*;
D77G T197I N283F D367N K370S S375H N401D S402Q F432V L435A T486V S531R Q595R D688A V821*S822*P823*D824*H825*G826*K827*K828*;
D77G T197I N283F D367N K370S N401D S402Q Y412F F432V L435A T486V S531R Q595R D688A V821*S822*P823*D824*H825*G826*K827*K828*;
D77G T197I N283F D367N K370S N401D S402Q F432V L435A T486V S531R A533I Q595R D688A V821*S822*P823*D824*H825*G826*K827*K828*;
D77G T197I N283F D367N K370S N401D S402Q F432V L435A T486V S531R L581F Q595R D688A V821*S822*P823*D824*H825*G826*K827*K828*;
D77G T197I N283F K370S N401D S402Q F432V L435A T486V S531R Q595R V665I V821*S822*P823*D824*H825*G826*K827*K828*;
D77G T197I N283F K370S N401D S402Q F432V L435A T486V S531R Q595R F700L V821*S822*P823*D824*H825*G826*K827*K828*;
D77G T197I N283F K370S N401D S402Q F432V L435A T486V S531R Q595R P709I V821*S822*P823*D824*H825*G826*K827*K828*;
D77G T197I N283F D367N K370S S375H N401D S402Q Y412F F432V L435A T486V S531R Q595R D688A V821*S822*P823*D824*H825*G826*K827*K828*;
D77G T197I N283F D367N K370S S375H N401D S402Q F432V L435A T486V S531R A533I Q595R D688A V821*S822*P823*D824*H825*G826*K827*K828*;
D77G T197I N283F D367N K370S S375H N401D S402Q Y412F F432V L435A T486V S531R A533I L581F Q595R D688A V821*S822*P823*D824*H825*G826*K827*K828*;
D77G T197I N283F H321V D367N K370S S375H N401D S402Q Y412F F432V Q434E L435A T486V S531R A533I L581F Q595R D688A V821*S822*P823*D824*H825*G826*K827*K828*;
D77G T197I N283F H321V D367N K370S S375H N401D S402Q Y412F F432V L435A T486V Q498R S531R A533I L581F Q595R D688A V821*S822*P823*D824*H825*G826*K827*K828*;
D77G T197I N283F H321V D367N K370S S375H N401D S402Q Y412F F432V L435A T486V S531R A533I L581F Q595R D688A F700L V821*S822*P823*D824*H825*G826*K827*K828*;
D77G T197I N283F H321V D367N K370S S375H N401D S402Q Y412F F432V Q434E L435A T486V Q498R S531R A533I L581F Q595R D688A F700L V821*S822*P823*D824*H825*G826*K827*K828*;
F17Y D77G T197I N283F K370S N401D S402Q F432V L435A T486V S531R Q595R V821*S822*P823*D824*H825*G826*K827*K828*;
D77G T197I N283F K370S N401D S402Q F432V L435A T486V S531R Q595R E804S V821*S822*P823*D824*H825*G826*K827*K828*;
D77G T197I N283F K370S N401D S402Q F432V L435A T486V S531R Q595R G811R V821*S822*P823*D824*H825*G826*K827*K828*;
D77G T197I N283F H321V K370S S375H N401D S402Q Y412F F432V Q434E L435A H479N T486V Q498R S531R A533I L581F Q595R D688A F700L V821*S822*P823*D824*H825*G826*K827*K828*;
D77G A190I T197I N283F K370S S375H N401D S402Q Y412F F432V Q434E L435A H479N T486V Q498R S531R A533I L581F Q595R D688A F700L V821*S822*P823*D824*H825*G826*K827*K828*;
D77G T197I T262V N283F K370S S375H N401D S402Q Y412F F432V Q434E L435A H479N T486V Q498R S531R A533I L581F Q595R D688A F700L V821*S822*P823*D824*H825*G826*K827*K828*; and
D77G A190I T197I N283F H321V D367N K370S S375H N401D S402Q Y412F F432V Q434E L435A T486V Q498R S531R A533I L581F Q595R D688A F700L V821*S822*P823*D824*H825*G826*K827*K828*.

The specific variants above may further comprise the substitutions N222P+Q252A+Q256R+N368G+N393A+Q431E+N610R+G624S+T631S+S632C, and optionally L432F. Particularly, the specific variants above may further comprise the substitutions N222P+Q252A+Q256R+N368G+N393A+Q431E+N610R+G624S+T631S+S632C+N20G+Y28K+H80Y+Q187R+E310A+D311K+Q387L+Q459G+D586S+E699R+S798R, and optionally L432F.

More particularly, the variant pullulanases may further comprise the deletions P30*+V31*+N32*, and optionally Q29G.

Even more particularly, the specific variants above may further comprise the substitutions N222P+Q252A+Q256R+N368G+N393A+Q431E++N610R+G624S+T631S+S632C+N20G+Y28K+H80Y+Q187R+E310A+D311K+Q387L+Q459G+D586S+E699R+S798R+Q29G+P30*+V31*+N32*+D57N+D58P+A195G+N202K+A345P+F456W+E560R, and optionally one, two three, four, five or six of L432F, N197T, M402S, N479H, I460V, I514V.

Most preferably the variants comprise the substitutions X370S+X432V, particularly K370S+L,F432V, and optionally X492A,S.

The pullulanase variants according to the invention have improved properties compared to the parent pullulanase. In particular, increased thermo-stability and/or increased thermo-activity. In a particular embodiment, the variant pullulanases have an increase in thermo-stability relative to a parent pullulanase, such as the parent disclosed in SEQ ID NO: 3 of at least 5%, at least 10%, at least 20%, at least 25%, at least 30%, at least 35%, at least 40%, at least 50%, at least 60%, at least 70%, at least 80%, at least 90%, or at least 100%, after enzymatic reaction of a maltodextrin substrate 2 hours at 85° C. or 30 min 91° C., and subsequent determination of digested maltodextrin fraction by PAHBAH assay at 55° C. Alternatively, the variant pullulanases have an increase in thermo-stability relative to a parent pullulanase, such as the parent disclosed in SEQ ID NO: 3, determined as increased melting (denaturing) temperature compared to the parent pullulanase disclosed in SEQ ID NO: 3 using TSA assay, of at least 0.3 degrees C., at least 0.4 degrees C., at least 0.5 degrees C., at least 0.6 degrees C., at least 0.8 degrees C., at least 1.0 degrees C., at least 1.2 degrees C., at least 1.5 degrees C., at least 2.0 degrees C., at least 2.5 degrees C., at least 3.0 degrees C., at least 3.5 degrees C., at least 4.0 degrees C., at least 4.5 degrees C., at least 5.0 degrees C.

### Polynucleotides

The present invention also relates to polynucleotides encoding a variant of the present invention.

### Nucleic Acid Constructs

The present invention also relates to nucleic acid constructs comprising a polynucleotide encoding a variant of the present invention operably linked to one or more control sequences that direct the expression of the coding sequence in a suitable host cell under conditions compatible with the control sequences.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide which is recognized by a host cell for expression of the polynucleotide. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator sequence is operably linked to the 3′-terminus of the polynucleotide encoding the variant. Any terminator that is functional in the host cell may be used.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader sequence is operably linked to the 5′-terminus of the polynucleotide encoding the variant. Any leader that is functional in the host cell may be used.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the variant-encoding sequence and, when transcribed, is recognized by the host cell as a signal to add polyadenosine residues to transcribed mRNA. Any polyadenylation sequence that is functional in the host cell may be used.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of the variant and the signal peptide sequence is positioned next to the N-terminus of the propeptide sequence.

It may also be desirable to add regulatory sequences that regulate expression of the variant relative to the growth of the host cell. Examples of regulatory systems are those that cause expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In yeast, the ADH2 system or GAL1 system may be used.

### Expression Vectors

The present invention also relates to recombinant expression vectors comprising a polynucleotide encoding a variant of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal clement, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD) (nitrate reductase), pyrG (orotidine-5′-phosphate decarboxylase), sC (sulfate adenyltransferase), and trpC (anthranilate synthase), as well as equivalents thereof.

The vector preferably contains an element(s) that permits integration of the vector into the host cell's genome or autonomous replication of the vector in the cell independent of the genome.

For integration into the host cell genome. the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMß1 permitting replication in Bacillus.

Examples of origins of replication for use in a yeast host cell are the 2 micron origin of replication, ARS1, ARS4, the combination of ARS1 and CEN3, and the combination of ARS4 and CEN6.

More than one copy of a polynucleotide of the present invention may be inserted into a host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide encoding a variant of the present invention operably linked to one or more control sequences that direct the production of a variant of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the variant and its source.

The host cell may be any cell useful in the recombinant production of a variant, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Hyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Kochler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397), or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804) or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a variant, comprising: (a) cultivating a host cell of the present invention under conditions suitable for expression of the variant; and (b) recovering the variant.

The host cells are cultivated in a nutrient medium suitable for production of the variant using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variant may be detected using methods known in the art. For example, an enzyme assay may be used to determine the activity of the variant. See the Assay section for suitable pullulanase activity assays.

The variant may be recovered using methods known in the art. For example, the variant may be recovered from the nutrient medium by conventional procedures including, but not limited to, collection, centrifugation, filtration, extraction, spray-drying, evaporation, or precipitation.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

In an alternative aspect, the variant is not recovered, but rather a host cell of the present invention expressing the variant is used as a source of the variant. In a particular embodiment, the pullulanase variants of the invention are produced in a yeast host cell which is also used as a fermenting organism in the processes of the invention, e.g., in SSF. Particularly, the yeast is a Saccharomyces cerevisiae.

### Fermentation Broth Formulations or Cell Compositions

The present invention also relates to a fermentation broth formulation or a cell composition comprising a polypeptide of the present invention. The fermentation broth product further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Enzyme Compositions

The present invention also relates to compositions comprising a pullulanase variant of the invention and a suitable stabilizer.

The compositions may comprise the pullulanase variant as the major enzymatic component, e.g., a mono-component composition. Alternatively, the compositions may comprise multiple enzymatic activities, such as one or more (e.g., several) enzymes selected from the group consisting of alpha-amylase, glucoamylase, beta-amylase, protease.

The compositions may be prepared in accordance with methods known in the art and may be in the form of a liquid or a dry composition. The compositions may be stabilized in accordance with methods known in the art.

Examples are given below of preferred uses of the compositions of the present invention. In a particular embodiment the composition further comprises an alpha-amylase.

The alpha-amylase is preferably a bacterial acid stable alpha-amylase. Particularly the alpha-amylase is from an Exiguobacterium sp. or a Bacillus sp. such as e.g., Bacillus stearothermophilus or Bacillus licheniformis.

In an embodiment the alpha-amylase is from the genus Bacillus, such as a strain of Bacillus stearothermophilus, in particular a variant of a Bacillus stearothermophilus alpha-amylase, such as the one shown in SEQ ID NO: 3 in WO 99/019467 or SEQ ID NO: 6 herein.

In an embodiment the Bacillus stearothermophilus alpha-amylase has a double deletion of two amino acids in the region from position 179 to 182, more particularly a double deletion at positions I181+G182, R179+G180, G180+I181, R179+I181, or G180+G182, preferably I181+G182, and optionally a N193F substitution, (using SEQ ID NO: 6 for numbering).

In an embodiment the Bacillus stearothermophilus alpha-amylase has a substitution at position S242, preferably S242Q substitution.

In an embodiment the Bacillus stearothermophilus alpha-amylase has a substitution at position E188, preferably E188P substitution.

In an embodiment the alpha-amylase is selected from the group of Bacillus stearothermophilus alpha-amylase variants with the following mutations in addition to a double deletion in the region from position 179 to 182, particularly I181*+G182* and optionally N193F.

In an embodiment the alpha-amylase is selected from the group of Bacillus stearothermophilus alpha-amylase variants with the following mutations:

$$I {181}^{*} + G {182}^{*} + N 193 F + E 129 V + K 177 L + R 179 E ;$$

$$I {181}^{*} + G {182}^{*} + N 193 F + V 59 A + Q 89 R + E 129 V + K 177 L + R 179 E + H 208 Y + K 220 P + N 224 L + Q 254 S ;$$

$$I {181}^{*} + G {182}^{*} + N 193 F + V 59 A Q 89 R + E 129 V + K 177 L + R 179 E + Q 254 S + M 284 V ;$$

$$I {181}^{*} + G {182}^{*} + N 193 F + V 59 A Q 89 R + E 129 V + K 177 L + R 179 S + Q 254 S + M 284 V ; and$$

$$I {181}^{*} + G {182}^{*} + N 193 F + E 129 V + K 177 L + R 179 E + K 220 P + N 224 L + S 242 Q + Q 254 S ( using SEQ ID NO : 6 for numbering ) .$$

In an embodiment the alpha-amylase variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 6.

It should be understood that when referring to Bacillus stearothermophilus alpha-amylase and variants thereof they are normally produced in truncated form. In particular, the truncation may be so that the Bacillus stearothermophilus alpha-amylase shown in SEQ ID NO: 3 in WO 99/19467 or SEQ ID NO: 6 herein, or variants thereof, are truncated in the C-terminal preferably to have around 490 amino acids, such as from 482-493 amino acids. Preferably the Bacillus stearothermophilus variant alpha-amylase is truncated, preferably after position 484 of SEQ ID NO: 6, particularly after position 485, particularly after position 486, particularly after position 487, particularly after position 488, particularly after position 489, particularly after position 490, particularly after position 491, particularly after position 492, more particularly after position 493.

### Protease Present and/or Added During Liquefaction

In a preferred embodiment the enzyme composition of the invention, further comprises a protease.

According to the invention a thermostable protease may optionally be present and/or added during liquefaction together with a variant pullulanase of the invention and an alpha-amylase, such as a thermostable alpha-amylase.

Proteases are classified on the basis of their catalytic mechanism into the following groups: Serine proteases (S), Cysteine proteases (C), Aspartic proteases (A), Metallo proteases (M), and Unknown, or as yet unclassified, proteases (U), see Handbook of Proteolytic Enzymes, A. J. Barrett, N. D. Rawlings, J. F. Woessner (eds), Academic Press (1998), in particular the general introduction part.

In one embodiment the protease is a serine protease or a metallo-protease, such as an S8 serine protease.

In a preferred embodiment the thermostable protease used according to the invention is a “metallo protease” defined as a protease belonging to EC 3.4.24 (metalloendopeptidases); preferably EC 3.4.24.39 (acid metallo proteinases).

To determine whether a given protease is a metallo protease or not, reference is made to the above “Handbook of Proteolytic Enzymes” and the principles indicated therein. Such determination can be carried out for all types of proteases, be it naturally occurring or wild-type proteases; or genetically engineered or synthetic proteases.

Protease activity can be measured using any suitable assay, in which a substrate is employed, that includes peptide bonds relevant for the specificity of the protease in question. Assay-pH and assay-temperature are likewise to be adapted to the protease in question. Examples of assay-pH-values are pH 6, 7, 8, 9, 10, or 11. Examples of assay-temperatures are 30, 35, 37, 40, 45, 50, 55, 60, 65, 70 or 80° C.

Examples of protease substrates are casein, such as Azurine-Crosslinked Casein (AZCL-casein). Two protease assays are described below in the “Materials & Methods”-section, of which the so-called “AZCL-Casein Assay” is the preferred assay.

In an embodiment the thermostable protease has at least 20%, such as at least 30%, such as at least 40%, such as at least 50%, such as at least 60%, such as at least 70%, such as at least 80%, such as at least 90%, such as at least 95%, such as at least 100% of the protease activity of the Protease 196 variant or Protease Pfu determined by the AZCL-casein assay described in the “Materials & Methods” section.

There are no limitations on the origin of the protease used in a process of the invention as long as it fulfills the thermostability properties defined below.

The protease may be a variant of, e.g., a wild-type protease as long as the protease has the thermostability properties defined herein. In a preferred embodiment the thermostable protease is a variant of a metallo protease as defined above. In an embodiment the thermostable protease used in a process of the invention is of fungal origin, such as a fungal metallo protease, such as a fungal metallo protease derived from a strain of the genus Thermoascus, preferably a strain of Thermoascus aurantiacus, especially Thermoascus aurantiacus CGMCC No. 0670 (classified as EC 3.4.24.39).

In an embodiment the thermostable protease is a variant of the metallo protease disclosed as the mature part of SEQ ID NO: 2 disclosed in WO 2003/048353 or the mature part of SEQ ID NO: 1 in WO 2010/008841 or SEQ ID NO: 7 herein with the following mutations:
D79L+S87P+A112P+D142L;
D79L+S87P+D142L; or
A27K+D79L+Y82F+S87G+D104P+A112P+A126V+D142L, and wherein the protease variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 2 disclosed in WO 2003/048353 or the mature part of SEQ ID NO: 1 in WO 2010/008841 or SEQ ID NO: 7 herein.

The thermostable protease may also be derived from any bacterium as long as the protease has the thermo-stability properties defined according to the description.

In an embodiment the thermostable protease is derived from a strain of the bacterium Pyrococcus, such as a strain of Pyrococcus furiosus (pfu protease).

In an embodiment the protease is the one shown as SEQ ID NO: 1 in U.S. Pat. No. 6,358,726-B1 (Takara Shuzo Company) or SEQ ID NO: 8 herein.

In another embodiment the thermostable protease is one disclosed in SEQ ID NO: 8 herein or a protease having at least 80% identity, such as at least 85%, such as at least 90%, such as at least 95%, such as at least 96%, such as at least 97%, such as at least 98%, such as at least 99% identity to SEQ ID NO: 1 in U.S. Pat. No. 6,358,726-B1 or SEQ ID NO: 8 herein. The Pyroccus furiosus protease can be purchased from Takara Bio, Japan.

In another embodiment, the protease is selected from a Palaeococcus sp. S8 protease, particularly a Palaeococcus ferrophilus S8 protease shown as SEQ ID NO: 9 or a protease having at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 9.

In an embodiment the composition of the invention comprises:
i) a Bacillus stearothermophilus alpha-amylase, or a variant thereof;
ii) a variant pullulanase of the invention;
iii) optionally a protease; and
   wherein the ratio between alpha-amylase and protease is in the range from 1:1 and 1:50 (micro gram alpha-amylase:micro gram protease).

In an embodiment the ratio between alpha-amylase and protease is in the range between 1:3 and 1:40, such as around 1:4 (micro gram alpha-amylase:micro gram protease).

In an embodiment the ratio between alpha-amylase and pullulanase is between 1:1 and 1:10, such as around 1:2.5 or 1:5 (micro gram alpha-amylase:micro gram pullulanase).

The pullulanase may according to the invention be added in an effective amount which include the preferred amount of about 2-100 micro gram enzyme protein per gram DS, preferably 5-50 micro gram enzyme protein per gram DS. Pullulanase activity may be determined as NPUN. An Assay for determination of NPUN is described in the assay section.

### Carbohydrate-Source Generating Enzyme Present and/or Added During Liquefaction

According to the invention a carbohydrate-source generating enzyme, preferably a thermostable glucoamylase, is present and/or added during liquefaction together with a thermostable alpha-amylase and optionally a thermostable protease. As mentioned above a pullulanase may also be present and/or added during liquefaction step i).

The term “carbohydrate-source generating enzyme” includes any enzymes generating fermentable sugars. A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrates may be converted directly or indirectly to the desired fermentation product, preferably ethanol. According to the invention a mixture of carbohydrate-source generating enzymes may be used. Specific examples include glucoamylase (being glucose generators), beta-amylase and maltogenic amylase (being maltose generators).

In a preferred embodiment the carbohydrate-source generating enzyme is a thermostable glucoamylase. The carbohydrate-source generating enzyme, in particular thermostable glucoamylase, may be added together with or separately from the thermostable alpha-amylase and optionally the thermostable protease.

In an embodiment the carbohydrate-source generating enzyme, preferably a thermostable glucoamylase, has a Relative Activity heat stability at 85° C. of at least 20%, at least 30%, preferably at least 35%. In an embodiment the carbohydrate-generating enzyme is a glucoamylase having a relative activity at pH 4.5 of at least 80%, preferably at least 85%, preferably at least 90%, preferably at least 95%.

In a specific embodiment the carbohydrate-source generating enzyme is a thermostable glucoamylase, preferably of fungal origin, preferably a filamentous fungi, such as from a strain of the genus Penicillium, especially a strain of Penicillium oxalicum disclosed as SEQ ID NO: 2 in WO 2011/127802 and shown in SEQ ID NO: 16 herein.

In a preferred embodiment the carbohydrate-source generating enzyme is a variant of the Penicillium oxalicum glucoamylase disclosed as SEQ ID NO: 2 in WO 2011/127802 and shown in SEQ ID NOS: 16, having a K79V substitution. In another preferred embodiment the Penicillium oxalicum glucoamylase variant has a K79V substitution (using SEQ ID NO: 16 for numbering), and further comprises one of the following mutations:

$$P 11 F + T 65 A + Q 327 F ; or$$

$$P 2 N + P 4 S + P 11 F + T 65 A + Q 327 F ; or$$

$$P 11 F + D 26 C + K33C + T 65 A + Q 327 F ; or$$

$$P 2 N + P 4 S + P 11 F + T 65 A + Q 327 F + E 501 V + Y 504 T ; or$$

$$P 2 N + P 4 S + P 11 F + T 65 A + Q 327 F + E 501 V + Y 504 T ; or$$

$$P 11 F + T 65 A + Q 327 W + E 501 V + Y 504 T ,$$

and
wherein, the glucoamylase has at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 16.

The carbohydrate-source generating enzyme may, in one embodiment, be added in amounts from 0.1-100 micrograms EP/g, such as 0.5-50 micrograms EP/g, such as 1-25 micrograms EP/g, such as 2-12 micrograms EP/g DS.

### Processes of the Invention

The present invention relates to processes for producing fermentation products from starch-containing material. In particular the product is an alcohol, more particularly ethanol.

The inventors have found that an increased ethanol yield can be obtained when a pullulanase variant according to the invention, having increased thermo-activity and/or increased thermo-activity, is present or added during liquefaction together with at least an alpha-amylase.

### Process of Producing a Fermentation Product of the Invention

In a particular aspect the invention relates to a process for producing a syrup from starch-containing material comprising the steps of:
a) liquefying the starch-containing material at a temperature above the initial gelatinization temperature using an alpha-amylase and a variant pullulanase of the invention;
b) saccharifying using a glucoamylase.

In another particular aspect the invention relates to processes for producing fermentation products from starch-containing material comprising the steps of:
a) liquefying the starch-containing material at a temperature above the initial gelatinization temperature using: an alpha-amylase and a variant pullulanase of the invention;
b) saccharifying using a glucoamylase;
c) fermenting using a fermenting organism.

In a preferred embodiment the fermentation product is recovered after fermentation, such as by distillation. In an embodiment the fermentation product is an alcohol, preferably ethanol, especially fuel ethanol, potable ethanol and/or industrial ethanol.

### Alpha-Amylases Present and/or Added in Liquefaction

The alpha-amylase added during liquefaction step a) in a process of the invention may be any alpha-amylase. Preferred are bacterial alpha-amylases, which typically are stable at a temperature used in liquefaction.

In an embodiment the alpha-amylase is from a strain of the genus Exiguobacterium or Bacillus.

In a preferred embodiment the alpha-amylase is from a strain of Bacillus stearothermophilus, such as the sequence shown in SEQ ID NO: 3 in WO99/019467 or in SEQ ID NO: 6 herein. In an embodiment the alpha-amylase is the Bacillus stearothermophilus alpha-amylase shown in SEQ ID NO: 6 herein, such as one having at least 80%, such as at least 85%, such as at least 90%, such as at least 95%, such as at least 96%, such as at least 97%, such as at least 98%, such as at least 99% identity to SEQ ID NO: 6 herein.

In an embodiment the Bacillus stearothermophilus alpha-amylase or variant thereof is truncated, preferably at the C-terminal, preferably truncated to have around 491 amino acids, such as from 480-495 amino acids.

In an embodiment the Bacillus stearothermophilus alpha-amylase has a double deletion of two amino acids in the region from position 179 to 182, more particularly a double deletion at positions I181+G182, R179+G180, G180+I181, R179+I181, or G180+G182, preferably I181+G182, and optionally a N193F substitution, (using SEQ ID NO: 6 for numbering).

In an embodiment the Bacillus stearothermophilus alpha-amylase has a substitution at position S242, preferably S242Q substitution.

In an embodiment the Bacillus stearothermophilus alpha-amylase has a substitution at position E188, preferably E188P substitution.

In an embodiment the alpha-amylase is selected from the group of Bacillus stearothermophilus alpha-amylase variants with the following mutations in addition to a double deletion in the region from position 179 to 182, particularly I181*+G182*, and optionally N193F.

In a preferred embodiment the alpha-amylase is selected from the group of Bacillus stearothermophilus alpha-amylase variants:

$$I {181}^{*} + G {182}^{*} + N 193 F + E 129 V + K 177 L + R 179 E ;$$

$$I {181}^{*} + G {182}^{*} + N 193 F + V 59 A + Q 89 R + E 129 V + K 177 L + R 179 E + H 208 Y + K 220 P + N 224 L + Q 254 S ;$$

$$I {181}^{*} + G {182}^{*} + N 193 F + V 59 A Q 89 R + E 129 V + K 177 L + R 179 S + Q 254 S + M 284 V ; and$$

$$I {181}^{*} + G {182}^{*} + N 193 F + E 129 V + K 177 L + R 179 E + K 220 P + N 224 L + S 242 Q + Q 254 S ( using SEQ ID NO : 6 for numbering ) .$$

According to the invention the alpha-amylase variant has at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 6 herein.

The alpha-amylase may according to the invention be present and/or added in a concentration of 0.1-100 micro gram per gram DS, such as 0.5-50 micro gram per gram DS, such as 1-25 micro gram per gram DS, such as 1-10 micro gram per gram DS, such as 2-5 micro gram per gram DS.

In an embodiment the ratio between alpha-amylase and pullulanase is between 1:1 and 1:10, such as around 1:2.5 or 1:5 (micro gram alpha-amylase:micro gram pullulanase).

The pullulanase may according to the invention be added in an effective amount which include the preferred amount of about 2-100 micro gram enzyme protein per gram DS, preferably 5-50 micro gram enzyme protein per gram DS. Pullulanase activity may be determined as NPUN. An Assay for determination of NPUN is described in the assay section.

### Protease Present and/or Added During Liquefaction

In a preferred embodiment the processes of the invention, further comprises adding a protease in liquefaction.

According to the invention a thermostable protease may optionally be present and/or added during liquefaction together with a variant pullulanase of the invention and an alpha-amylase, such as a thermostable alpha-amylase.

For more details on suitable proteases see the composition section above.

In an preferred embodiment the thermostable protease is a variant of the metallo protease disclosed as the mature part of SEQ ID NO: 2 disclosed in WO 2003/048353 or the mature part of SEQ ID NO: 1 in WO 2010/008841 or SEQ ID NO: 7 herein with the following mutations:
D79L+S87P+A112P+D142L;
D79L+S87P+D142L; or
A27K+D79L+Y82F+S87G+D104P+A112P+A126V+D142L, and wherein, the protease variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 2 disclosed in WO 2003/048353 or the mature part of SEQ ID NO: 1 in WO 2010/008841 or SEQ ID NO: 7 herein.

The thermostable protease may also be derived from any bacterium as long as the protease has the thermo-stability properties defined according to the description.

In an embodiment the thermostable protease is derived from a strain of the bacterium Pyrococcus, such as a strain of Pyrococcus furiosus (pfu protease).

In an embodiment the protease is the one shown as SEQ ID NO: 1 in U.S. Pat. No. 6,358,726-B1 (Takara Shuzo Company) or SEQ ID NO: 8 herein.

In another embodiment the thermostable protease is one disclosed in SEQ ID NO: 8 herein or a protease having at least 80% identity, such as at least 85%, such as at least 90%, such as at least 95%, such as at least 96%, such as at least 97%, such as at least 98%, such as at least 99% identity to SEQ ID NO: 1 in U.S. Pat. No. 6,358,726-B1 or SEQ ID NO: 8 herein. The Pyroccus furiosus protease can be purchased from Takara Bio, Japan.

### Glucoamylase Present and/or Added in Saccharification and/or Fermentation

A glucoamylase is present and/or added in saccharification and/or fermentation, preferably simultaneous saccharification and fermentation (SSF), in a process of the invention (i.e., oil recovery process and fermentation product production process).

In an embodiment the glucoamylase present and/or added in saccharification and/or fermentation is of fungal origin, preferably from a stain of Aspergillus, preferably A. niger, A. awamori, or A. oryzae; or a strain of Trichoderma, preferably T. reesei; or a strain of Talaromyces, preferably T. emersonii or a strain of Trametes, preferably T. cingulata, or a strain of Pycnoporus, or a strain of Gloeophyllum, such as G. sepiarium or G. trabeum, or a strain of the Nigrofomes.

In an embodiment the glucoamylase is derived from Talaromyces, such as a strain of Talaromyces emersonii, such as the one shown in SEQ ID NO: 10 herein.

In an embodiment the glucoamylase is selected from the group consisting of:
(i) a glucoamylase comprising the polypeptide of SEQ ID NO: 10 herein;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 10 herein.

In an embodiment the glucoamylase is derived from a strain of the genus Pycnoporus, in particular a strain of Pycnoporus sanguineus described in WO 2011/066576 (SEQ ID NOs 2, 4 or 6), such as the one shown as SEQ ID NO: 4 in WO 2011/066576 or SEQ ID NO: 11 herein.

In an embodiment the glucoamylase is derived from a strain of the genus Gloeophyllum, such as a strain of Gloeophyllum sepiarium or Gloeophyllum trabeum, in particular a strain of Gloeophyllum as described in WO 2011/068803 (SEQ ID NO: 2, 4, 6, 8, 10, 12, 14 or 16). In a preferred embodiment the glucoamylase is the Gloeophyllum sepiarium shown in SEQ ID NO: 2 in WO 2011/068803 or SEQ ID NO: 12 herein.

In a preferred embodiment the glucoamylase is derived from Gloeophyllum sepiarium, such as the one shown in SEQ ID NO: 12 herein. In an embodiment the glucoamylase is selected from the group consisting of:
(i) a glucoamylase comprising the polypeptide of SEQ ID NO: 12 herein;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 12 herein.

In another embodiment the glucoamylase is derived from Gloeophyllum trabeum such as the one shown in SEQ ID NO: 13 herein. In an embodiment the glucoamylase is selected from the group consisting of:
(i) a glucoamylase comprising the polypeptide of SEQ ID NO: 13 herein;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 13 herein.

In an embodiment the glucoamylase is derived from a strain of the genus Trametes, in particular a strain of Trametes cingulata disclosed in WO 2006/069289, and herein as SEQ ID NO: 14.

Glucoamylases may in an embodiment be added to the saccharification and/or fermentation in an amount of 0.0001-20 AGU/g DS. preferably 0.001-10 AGU/g DS, especially between 0.01-5 AGU/g DS, such as 0.1-2 AGU/g DS.

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U, SPIRIZYME™ ULTRA, SPIRIZYME™ EXCEL and AMG™ E (from Novozymes A/S); OPTIDEX™ 300, GC480, GC417 (from DuPont.); AMIGASE™ and AMIGASE™ PLUS (from DSM); G-ZYME™ G900, G-ZYME™ and G990 ZR (from DuPont).

According to a preferred embodiment of the invention the glucoamylase is present and/or added in saccharification and/or fermentation in combination with an alpha-amylase. Examples of suitable alpha-amylase are described below.

### Alpha-Amylase Present and/or Added in Saccharification and/or Fermentation

In an embodiment an alpha-amylase is present and/or added in saccharification and/or fermentation in a process of the invention. In a preferred embodiment the alpha-amylase is of fungal or bacterial origin. In a preferred embodiment the alpha-amylase is a fungal acid stable alpha-amylase. A fungal acid stable alpha-amylase is an alpha-amylase that has activity in the pH range of 3.0 to 7.0 and preferably in the pH range from 3.5 to 6.5, including activity at a pH of about 4.0, 4.5, 5.0, 5.5, and 6.0.

In a preferred embodiment the alpha-amylase present and/or added in saccharification and/or fermentation is derived from a strain of the genus Rhizomucor, preferably a strain the Rhizomucor pusillus, such as one shown in SEQ ID NO: 3 in WO 2013/006756, such as a Rhizomucor pusillus alpha-amylase hybrid having an Aspergillus niger linker and starch-bonding domain, such as the one shown in SEQ ID NO: 15 herein, or a variant thereof.

In an embodiment the alpha-amylase present and/or added in saccharification and/or fermentation is selected from the group consisting of:
(i) an alpha-amylase comprising the polypeptide of SEQ ID NO: 15 herein;
(ii) an alpha-amylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 15 herein.

In a preferred embodiment the alpha-amylase is a variant of the alpha-amylase shown in SEQ ID NO: 15 having at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C (using SEQ ID NO: 15 for numbering).

In an embodiment the alpha-amylase is derived from a Rhizomucor pusillus with an Aspergillus niger glucoamylase linker and starch-binding domain (SBD), preferably disclosed as SEQ ID NO: 15 herein, preferably having one or more of the following substitutions: G128D, D143N, preferably G128D+D143N (using SEQ ID NO: 15 for numbering).

In an embodiment the alpha-amylase variant present and/or added in saccharification and/or fermentation has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 15 herein.

In a preferred embodiment the ratio between glucoamylase and alpha-amylase present and/or added during saccharification and/or fermentation may preferably be in the range from 500:1 to 1:1, such as from 250:1 to 1:1, such as from 100:1 to 1:1, such as from 100:2 to 100:50, such as from 100:3 to 100:70.

### Further Aspects of Processes of the Invention

Prior to liquefaction step a), processes of the invention, may comprise the steps of:
i) reducing the particle size of the starch-containing material, preferably by dry milling;
ii) forming a slurry comprising the starch-containing material and water.

In an embodiment at least 50%, preferably at least 70%, more preferably at least 80%, especially at least 90% of the starch-containing material fit through a sieve with #6 screen.

In an embodiment the pH during liquefaction is between above 4.5-6.5, such as 4.5-5.0, such as around 4.8, or a pH between 5.0-6.2, such as 5.0-6.0, such as between 5.0-5.5, such as around 5.2, such as around 5.4, such as around 5.6, such as around 5.8.

In an embodiment the temperature during liquefaction is above the initial gelatinization temperature, preferably in the range from 70-100° C., such as between 75-95° C., such as between 75-90° C., preferably between 80-90° C., especially around 85° C.

In an embodiment a jet-cooking step is carried out before liquefaction in step a). In an embodiment the jet-cooking is carried out at a temperature between 110-145° C., preferably 120-140° C., such as 125-135° C., preferably around 130° C. for about 1-15 minutes, preferably for about 3-10 minutes, especially around about 5 minutes.

In a preferred embodiment saccharification and fermentation is carried out sequentially or simultaneously.

In an embodiment saccharification is carried out at a temperature from 20-75° C., preferably from 40-70° C., such as around 60° C., and at a pH between 4 and 5.

In an embodiment fermentation or simultaneous saccharification and fermentation (SSF) is carried out carried out at a temperature from 25° C. to 40° C., such as from 28° C. to 35° C., such as from 30° C. to 34° C., preferably around about 32° C. In an embodiment fermentation is ongoing for 6 to 120 hours, in particular 24 to 96 hours.

In a preferred embodiment the fermentation product is recovered after fermentation, such as by distillation.

In an embodiment the fermentation product is an alcohol, preferably ethanol, especially fuel ethanol, potable ethanol and/or industrial ethanol.

In an embodiment the starch-containing starting material is whole grains. In an embodiment the starch-containing material is selected from the group of corn, wheat, barley, rye, milo, sago, cassava, manioc, tapioca, sorghum, rice, and potatoes.

In an embodiment the fermenting organism is yeast, preferably a strain of Saccharomyces, especially a strain of Saccharomyces cerevisae.

In an embodiment the temperature in step (a) is above the initial gelatinization temperature, such as at a temperature between 80-90° C., such as around 85° C.

In an embodiment a process of the invention further comprises a pre-saccharification step, before saccharification step b), carried out for 40-90 minutes at a temperature between 30-65° C. In an embodiment saccharification is carried out at a temperature from 20-75° C., preferably from 40-70° C., such as around 60° C., and at a pH between 4 and 5. In an embodiment fermentation step c) or simultaneous saccharification and fermentation (SSF) (i.e., steps b) and c)) are carried out carried out at a temperature from 25° C. to 40° C., such as from 28° C. to 35° C., such as from 30° C. to 34° C., preferably around about 32° C. In an embodiment the fermentation step c) or simultaneous saccharification and fermentation (SSF) (i.e., steps b) and c)) are ongoing for 6 to 120 hours, in particular 24 to 96 hours.

In an embodiment the fermentation product is recovered by distillation.

### Fermentation Medium

The environment in which fermentation is carried out is often referred to as the “fermentation media” or “fermentation medium”. The fermentation medium includes the fermentation substrate, that is, the carbohydrate source that is metabolized by the fermenting organism. According to the invention the fermentation medium may comprise nutrients and growth stimulator(s) for the fermenting organism(s). Nutrient and growth stimulators are widely used in the art of fermentation and include nitrogen sources, such as ammonia; urea, vitamins and minerals, or combinations thereof.

### Fermenting Organisms

The term “fermenting organism” refers to any organism, including bacterial and fungal organisms, especially yeast, suitable for use in a fermentation process and capable of producing the desired fermentation product. Especially suitable fermenting organisms are able to ferment, i.e., convert, sugars, such as glucose or maltose, directly or indirectly into the desired fermentation product, such as ethanol. Examples of fermenting organisms include fungal organisms, such as yeast. Preferred yeast includes strains of Saccharomyces spp., in particular, Saccharomyces cerevisiae.

Suitable concentrations of the viable fermenting organism during fermentation, such as SSF, are well known in the art or can easily be determined by the skilled person in the art. In one embodiment the fermenting organism, such as ethanol fermenting yeast, (e.g., Saccharomyces cerevisiae) is added to the fermentation medium so that the viable fermenting organism, such as yeast, count per mL of fermentation medium is in the range from 10⁵ to 10¹², preferably from 10⁷ to 10¹⁰, especially about 5×10⁷.

Examples of commercially available yeast includes, e.g., RED STAR™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

### Starch-Containing Materials

Any suitable starch-containing material may be used according to the present invention. The starting material is generally selected based on the desired fermentation product. Examples of starch-containing materials, suitable for use in a process of the invention, include whole grains, corn, wheat, barley, rye, milo, sago, cassava, tapioca, sorghum, rice, peas, beans, or sweet potatoes, or mixtures thereof or starches derived therefrom, or cereals. Contemplated are also waxy and non-waxy types of corn and barley. In a preferred embodiment the starch-containing material, used for ethanol production according to the invention, is corn or wheat.

### Fermentation Products

The term “fermentation product” means a product produced by a process including a fermentation step using a fermenting organism. Fermentation products contemplated according to the invention include alcohols (e.g., ethanol, methanol, butanol; polyols such as glycerol, sorbitol and inositol); organic acids (e.g., citric acid, acetic acid, itaconic acid, lactic acid, succinic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferably processes of the invention are used for producing an alcohol, such as ethanol. The fermentation product, such as ethanol, obtained according to the invention, may be used as fuel, which is typically blended with gasoline. However, in the case of ethanol it may also be used as potable ethanol.

### Recovery of Fermentation Products

Subsequent to fermentation, or SSF, the fermentation product may be separated from the fermentation medium. The slurry may be distilled to extract the desired fermentation product (e.g., ethanol). Alternatively the desired fermentation product may be extracted from the fermentation medium by micro or membrane filtration techniques. The fermentation product may also be recovered by stripping or other method well known in the art.

The invention is further summarized in the following numbered paragraphs:

1. A variant pullulanase, having increased thermo-stability and/or increased thermo-activity compared to a parent pullulanase, comprising a substitution at least at one position selected from a position corresponding to positions 432, 486, 370, 17, 77, 103, 106, 107, 190, 196, 197, 262, 279, 283, 321, 367, 375, 382, 399, 401, 402, 411, 412, 434, 435, 443, 446, 459, 460, 479, 490, 498, 514, 529, 531, 533, 541, 545, 581, 583, 595, 649, 665, 688, 700, 709, 804, and 811 of SEQ ID NO: 1, and optionally a deletion of one or more, e.g., all amino acids at positions 821, 822, 823, 824, 825, 826, 827, and 828, wherein the variant has pullulanase activity, and wherein the variant has at least 70%, at least 75%, at least 80%, at least 85%, at least 90%, at least 95%, at least 96%, at least 97%, at least 98%, or at least 99%, but less than 100% sequence identity to a parent alpha amylase selected from the group consisting of SEQ ID NO: 1, SEQ ID NO: 3, SEQ ID NO: 4, SEQ ID NO: 5.

2. The variant pullulanase of paragraph 1, comprising a substitution at a position corresponding to position 432 of SEQ ID NO: 1, wherein the variant pullulanase comprises valine in position 432 using SEQ ID NO: 1 for numbering, particularly a F432V substitution, wherein the variant has pullulanase activity, and wherein the variant has at least 70%, at least 75%, at least 80%, at least 85%, at least 90%, at least 95%, at least 96%, at least 97%, at least 98%, or at least 99%, but less than 100% sequence identity to a parent alpha amylase selected from the group consisting of SEQ ID NO: 1. SEQ ID NO: 3, SEQ ID NO: 4, SEQ ID NO: 5.

3. The variant pullulanase of paragraph 1, comprising a substitution at a position corresponding to position 486 of SEQ ID NO: 1, wherein the variant pullulanase comprises alanine or valine in position 486 using SEQ ID NO: 1 for numbering, particularly a T486A, V substitution, wherein the variant has pullulanase activity, and wherein the variant has at least 70%, at least 75%, at least 80%, at least 85%, at least 90%, at least 95%, at least 96%, at least 97%, at least 98%, or at least 99%, but less than 100% sequence identity to a parent alpha amylase selected from the group consisting of SEQ ID NO: 1, SEQ ID NO: 3, SEQ ID NO: 4, SEQ ID NO: 5.

4. The variant pullulanase of paragraph 1, comprising a substitution at a position corresponding to position 370 of SEQ ID NO: 1, wherein the variant pullulanase comprises serine in position 370 using SEQ ID NO: 1 for numbering, particularly a K370S substitution, wherein the variant has pullulanase activity, and wherein the variant has at least 70%, at least 75%, at least 80%, at least 85%, at least 90%, at least 95%, at least 96%, at least 97%, at least 98%, or at least 99%, but less than 100% sequence identity to a parent alpha amylase selected from the group consisting of SEQ ID NO: 1, SEQ ID NO: 3, SEQ ID NO: 4, SEQ ID NO: 5.

5. The variant pullulanase of any of the preceding paragraphs, wherein the substitutions are selected from the group consisting of: K370S, F17Y, D77G, S103K, Q106W, A107D, A190I, V196T,C, T197I, T262V, Q279R, N283F, H321V, D367G,N, S375H, N382T, Q399N, N401D, S402Q, N411L, Y412F, F432V, Q434E, L435A, R443G, I446V, G459E, V460E, H479N, T486A,V, I490L, Q498R, V514A, T529L, S531R, A533I, N541D, A545I, L581F, N583D, Q595R, D649A, V665I, D688A, F700L, P709I, E804S, and G811R.

6. The variant pullulanase according to any of paragraphs 1-5, wherein thermo-stability is determined as relative activity after heat stress for 30 min at two different temperatures, e.g., selected in the range from 60° C.-90° C., e.g., 70° C.-87° C., and subsequently assayed at 60-80° C., e.g., 70° C., using PHADEBAS assay.

7. The variant pullulanase according to any of paragraphs 1-5, wherein thermo-stability is determined as increased melting (denaturing) temperature compared to the parent pullulanase using TSA assay.

8. The variant pullulanase according to any of paragraphs 1-5, wherein thermo-activity is determined as relative activity determined at two different temperatures selected from the range of 70-86° C. using PHADEBAS assay.

9. The variant pullulanase according to any of the preceding paragraphs, wherein the variant comprises combinations of substitutions and/or deletions selected from the group consisting of:
Q279R+K370S;
H321E+K370S;
K370S+V821*+S822*+P823*+D824*+H825*+G826*+K827*+K828*;
K370S+Q399N;
K370S+N401D;
K370S+F432V;
V196T+K370S;
V196C+K370S;
T197I+K370S;
K370S+V460E;
K370S+T486A;
K370S+T486V;
K370S+I490L;
K370S+V514A;
K370S+T529L;
K370S+S531R;
K370S+Q595R;
H321E+K370S+F432V;
K370S+F432V+V821*+S822*+P823*+D824*+H825*+G826*+K827*+K828*;
K370S+Q399N+F432V;
K370S+N401D+F432V;
V196T+K370S+F432V;
V196C+K370S+F432V;
T197I+K370S+F432V;
K370S+F432V+G459E;
K370S+F432V+T486A;
K370S+F432V+T486V;
V196T+K370S+F432V+T486A;
T197I+K370S+F432V+T486A;
V196T+T197I+K370S+F432V+T486A;
K370S+Q399N+F432V+T486A;
K370S+N401D+F432V+T486A;
K370S+Q399N+N401D+F432V+T486A;
K370S+S531R+F432V+T486A;
K370S+Q595R+F432V+T486A;
K370S+F432V+T486A+S531R+Q595R;
V196T+T197I+K370S+Q399N+N401D+F432V+T486A;
V196T+T197I+K370S+Q399N+N401D+F432V+T486A+Q595R;
T197I+K370S+N401D+F432V+T486A+S531R+Q595R;
T197I+K370S+N401D+F432V+T486A+S531R+Q595R+V821*+S822*+P823*+D824*+H825*+G826*+K827*+K828*;
T197I+K370S+Q399N+N401D+F432V+T486A+Q595R;
S103K+T197I+K370S+N401D+F432V+T486A+S531R+Q595R;
V196T+T197I+K370S+Q399N+N401D+F432V+T486V+Q595R;
T197I+K370S+N401D+F432V+T486V+S531R+Q595R;
V196T+T197I+K370S+Q399N+N401D+F432V+T486A+S531R+Q595R;
D77G+T197I+K370S+N401D+F432V+T486V+S531R+Q595R;
T197I+K370S+N401D+S402Q+F432V+T486V+S531R+Q595R;
T197I+N283F+K370S+N401D+F432V+T486V+S531R+Q595R;
T197I+N283F+K370S+N401D+S402Q+F432V+T486V+S531R+Q595R;
T197I+N283F+K370S+N401D+F432V+L435A+T486V+S531R+Q595R;
D77G+T197I+N283F+K370S+N401D+S402Q+F432V+T486V+S531R+Q595R;
T197I+N283F+K370S+N401D+S402Q+F432V+L435A+T486V+S531R+Q595R;
D77G+T197I+N283F+K370S+N401D+S402Q+F432V+L435A+T486V+S531R+Q595R;
T197I+N283F+K370S+N401D+S402Q+F432V+L435A+T486V+S531R+Q595R+D649A;
D77G+T197I+N283F+K370S+N401D+S402Q+F432V+L435A+T486V+S531R+Q595R+D649A;
D77G+Q106W+T197I+N283F+K370S+N401D+S402Q+F432V+L435A+T486V+S531R+Q595R;
D77G+A107D+T197I+N283F+K370S+N401D+S402Q+F432V+L435A+T486V+S531R+Q595R;
D77G+V196C+T197I+N283F+K370S+N401D+S402Q+F432V+L435A+T486V+S531R+Q595R;
D77G+T197I+N283F+K370S+N401D+S402Q+F432V+L435A+T486V+S531R+N583D+Q595R;
D77G+T197I+N283F+K370S+N401D+S402Q+F432V+L435A+T486V+S531R+Q595R+V821*+S822*+P823*+D824*+H825*+G826*+K827*+K828*;
D77G+T197I+N283F+K370S+N401D+S402Q+F432V+L435A+T486V+S531R+Q595R+D688A+V821*+S822*+P823*+D824*+H825*+G826*+K827*+K828*;
D77G+T197I+N283F+D367G+K370S+N401D+S402Q+F432V+L435A+T486V+S531R+Q595R+V821*+S822*+P823*+D824*+H825*+G826*+K827*+K828*;
D77G+T197I+N283F+D367N+K370S+N401D+S402Q+F432V+L435A+T486V+S531R+Q595R+V821*+S822*+P823*+D824*+H825*+G826*+K827*+K828*;
D77G+T197I+N283F+K370S+S375H+N401D+S402Q+F432V+L435A+T486V+S531R+Q595R+V821*+S822*+P823*+D824*+H825*+G826*+K827*+K828*;
D77G+T197I+N283F+K370S+N382T+N401D+S402Q+F432V+L435A+T486V+S531R+Q595R+V821*+S822*+P823*+D824*+H825*+G826*+K827*+K828*;
D77G+T197I+N283F+K370S+N401D+S402Q+N411L+F432V+L435A+T486V+S531R+Q595R+V821*+S822*+P823*+D824*+H825*+G826*+K827*+K828*;
D77G+T197I+N283F+K370S+N401D+S402Q+Y412F+F432V+L435A+T486V+S531R+Q595R+V821*+S822*+P823*+D824*+H825*+G826*+K827*+K828*;
D77G+T197I+N283F+K370S+N401D+S402Q+F432V+Q434E+L435A+T486V+S531R+Q595R+V821*+S822*+P823*+D824*+H825*+G826*+K827*+K828*;
D77G+T197I+N283F+K370S+N401D+S402Q+F432V+L435A+R443G+T486V+S531R+Q595R+V821*+S822*+P823*+D824*+H825*+G826*+K827*+K828*;
D77G+T197I+N283F+K370S+N401D+S402Q+F432V+L435A+I446V+T486V+S531R+Q595R+V821*+S822*+P823*+D824*+H825*+G826*+K827*+K828*;
D77G+T197I+N283F+K370S+N401D+S402Q+F432V+L435A+T486V+Q498R+S531R+Q595R+V821*+S822*+P823*+D824*+H825*+G826*+K827*+K828*;
D77G+T197I+N283F+K370S+N401D+S402Q+F432V+L435A+T486V+S531R+A533I+Q595R+V821*+S822*+P823*+D824*+H825*+G826*+K827*+K828*;
D77G+T197I+N283F+K370S+N401D+S402Q+F432V+L435A+T486V+S531R+N541D+Q595R+V821*+S822*+P823*+D824*+H825*+G826*+K827*+K828*;
D77G+T197I+N283F+K370S+N401D+S402Q+F432V+L435A+T486V+S531R+A545I+Q595R+V821*+S822*+P823*+D824*+H825*+G826*+K827*+K828*;
D77G+T197I+N283F+K370S+N401D+S402Q+F432V+L435A+T486V+S531R+L581F+Q595R+V821*+S822*+P823*+D824*+H825*+G826*+K827*+K828*;
D77G+T197I+N283F+K370S+N401D+S402Q+F432V+L435A+H479N+T486V+S531R+Q595R+D688A+V821*+S822*+P823*+D824*+H825*+G826*+K827*+K828*;
D77G+T197I+N283F+D367N+K370S+N401D+S402Q+F432V+L435A+T486V+S531R+Q595R+D688A+V821*+S822*+P823*+D824*+H825*+G826*+K827*+K828*;
D77G+T197I+N283F+K370S+S375H+N401D+S402Q+F432V+L435A+T486V+S531R+Q595R+D688A+V821*+S822*+P823*+D824*+H825*+G826*+K827*+K828*;
D77G+T197I+N283F+K370S+N401D+S402Q+Y412F+F432V+L435A+T486V+S531R+Q595R+D688A+V821*+S822*+P823*+D824*+H825*+G826*+K827*+K828*;
D77G+T197I+N283F+K370S+N401D+S402Q+F432V+L435A+T486V+S531R+A533I+Q595R+D688A+V821*+S822*+P823*+D824*+H825*+G826*+K827*+K828*;
D77G+T197I+N283F+K370S+N401D+S402Q+F432V+L435A+T486V+S531R+L581F+Q595R+D688A+V821*+S822*+P823*+D824*+H825*+G826*+K827*+K828*;
D77G T197I N283F D367N K370S S375H N401D S402Q F432V L435A T486V S531R Q595R D688A V821*S822*P823*D824*H825*G826*K827*K828*;
D77G T197I N283F D367N K370S N401D S402Q Y412F F432V L435A T486V S531R Q595R D688A V821*S822*P823*D824*H825*G826*K827*K828*;
D77G T197I N283F D367N K370S N401D S402Q F432V L435A T486V S531R A533I Q595R D688A V821*S822*P823*D824*H825*G826*K827*K828*;
D77G T197I N283F D367N K370S N401D S402Q F432V L435A T486V S531R L581F Q595R D688A V821*S822*P823*D824*H825*G826*K827*K828*;
D77G T197I N283F K370S N401D S402Q F432V L435A T486V S531R Q595R V665I V821*S822*P823*D824*H825*G826*K827*K828*;
D77G T197I N283F K370S N401D S402Q F432V L435A T486V S531R Q595R F700L V821*S822*P823*D824*H825*G826*K827*K828*;
D77G T197I N283F K370S N401D S402Q F432V L435A T486V S531R Q595R P709I V821*S822*P823*D824*H825*G826*K827*K828*;
D77G T197I N283F D367N K370S S375H N401D S402Q Y412F F432V L435A T486V S531R Q595R D688A V821*S822*P823*D824*H825*G826*K827*K828*;
D77G T197I N283F D367N K370S S375H N401D S402Q F432V L435A T486V S531R A533I Q595R D688A V821*S822*P823*D824*H825*G826*K827*K828*;
D77G T197I N283F D367N K370S S375H N401D S402Q Y412F F432V L435A T486V S531R A533I L581F Q595R D688A V821*S822*P823*D824*H825*G826*K827*K828*;
D77G T197I N283F H321V D367N K370S S375H N401D S402Q Y412F F432V Q434E L435A T486V S531R A533I L581F Q595R D688A V821*S822*P823*D824*H825*G826*K827*K828*;
D77G T197I N283F H321V D367N K370S S375H N401D S402Q Y412F F432V L435A T486V Q498R S531R A533I L581F Q595R D688A V821*S822*P823*D824*H825*G826*K827*K828*;
D77G T197I N283F H321V D367N K370S S375H N401D S402Q Y412F F432V L435A T486V S531R A533I L581F Q595R D688A F700L V821*S822*P823*D824*H825*G826*K827*K828*;
D77G T197I N283F H321V D367N K370S S375H N401D S402Q Y412F F432V Q434E L435A T486V Q498R S531R A533I L581F Q595R D688A F700L V821*S822*P823*D824*H825*G826*K827*K828*;
F17Y D77G T197I N283F K370S N401D S402Q F432V L435A T486V S531R Q595R V821*S822*P823*D824*H825*G826*K827*K828*;
D77G T197I N283F K370S N401D S402Q F432V L435A T486V S531R Q595R E804S V821*S822*P823*D824*H825*G826*K827*K828*;
D77G T197I N283F K370S N401D S402Q F432V L435A T486V S531R Q595R G811R V821*S822*P823*D824*H825*G826*K827*K828*;
D77G T197I N283F H321V K370S S375H N401D S402Q Y412F F432V Q434E L435A H479N T486V Q498R S531R A533I L581F Q595R D688A F700L V821*S822*P823*D824*H825*G826*K827*K828*;
D77G A190I T197I N283F K370S S375H N401D S402Q Y412F F432V Q434E L435A H479N T486V Q498R S531R A533I L581F Q595R D688A F700L V821*S822*P823*D824*H825*G826*K827*K828*;
D77G T197I T262V N283F K370S S375H N401D S402Q Y412F F432V Q434E L435A H479N T486V Q498R S531R A533I L581F Q595R D688A F700L V821*S822*P823*D824*H825*G826*K827*K828*; and
D77G A190I T197I N283F H321V D367N K370S S375H N401D S402Q Y412F F432V Q434E L435A T486V Q498R S531R A533I L581F Q595R D688A F700L V821*S822*P823*D824*H825*G826*K827*K828*.

10. The variant pullulanase of any of the preceding paragraphs, comprising the substitutions N222P+Q252A+Q256R+N368G+N393A+Q431E+N610R+G624S+T631S+S632C, and optionally L432F.

11. The variant pullulanase of any of the preceding paragraphs, comprising the substitutions N222P+Q252A+Q256R+N368G+N393A+Q431E+N610R+G624S+T631S+S632C+N20G+Y28K+H80Y+Q187R+E310A+D311K+Q387L+Q459G+D586S+E699R+S798R, and optionally L432F.

12. The variant pullulanase of any of the preceding paragraphs, comprising the deletions P30*+V31*+N32*, and optionally Q29G.

13. The variant pullulanase of any of the preceding paragraphs, comprising the modifications Q29G+P30*+V31*+N32*+D57N+D58P+A195G+N202K+A345P+F456W+E560R, and optionally one, two, three, four or five of N197T, M402S, I460V, N479H, I514V.

14. The variant pullulanase of any of the preceding paragraphs, comprising the modifications N222P+Q252A+Q256R+N368G+N393A+Q431E++N610R+G624S+T631S+S632C+N20G+Y28K+H80Y+Q187R+E310A+D311K+Q387L+Q459G+D586S+E699R+S798R+Q29G+P30*+V31*+N32*+D57N+D58P+A195G+N202K+A345P+F456W+E560R, and optionally one, two, three, four, five or six of L432F, N197T, M402S, N479H, I460V, I514V.

15. The variant of any of the preceding paragraphs, wherein the variant comprises the substitutions X370S+X432V, particularly K370S+L,F432V, and optionally X492A,S.

16. The variant according to any of claims 1-15, wherein the variants have an increase in thermo-activity relative to a parent pullulanase, e.g., the pullulanase disclosed as SEQ ID NO: 3, of at least 5%, at least 10%, at least 20%, at least 25%, at least 30%, at least 35%, at least 40%, at least 50%, at least 60%, at least 70%, at least 80%, at least 90%, at least 100%, after enzymatic reaction of maltodextrin 2 hours at 85° C. or 30 min 91° C., and subsequent determination of digested maltodextrin fraction by PAHBAH assay at 55° C.

17. The variants of any of the preceding paragraphs, wherein the increase in thermo-stability determined as increased melting (denaturing) temperature compared to the parent pullulanase disclosed in SEQ ID NO: 3 using TSA assay is at least 0.3 degrees C., at least 0.4 degrees C., at least 0.5 degrees C., at least 0.6 degrees C., at least 0.8 degrees C., at least 1.0 degrees C., at least 1.2 degrees C., at least 1.5 degrees C., at least 2.0 degrees C., at least 2.5 degrees C., at least 3.0 degrees C., at least 3.5 degrees C., at least 4.0 degrees C., at least 4.5 degrees C., at least 5.0 degrees C.

18. A polynucleotide encoding the variant pullulanase of any of the paragraphs 1-17.

19. A nucleic acid construct or expression vector comprising the polynucleotide of paragraph 18 operably linked to one or more control sequences that direct the production of the polypeptide in an expression host.

20. A recombinant host cell comprising the polynucleotide of paragraph 18 operably linked to one or more control sequences that direct the production of the polypeptide.

21. A composition comprising the variant pullulanase of any of paragraphs 1-17 and a stabilizer.

22. The composition of paragraph 21, comprising an alpha-amylase.

23. The composition of paragraph 22, wherein the alpha-amylase is a bacterial alpha-amylase, particularly derived from Bacillus or Exiguobacterium species, such as, e.g., Bacillus licheniformis or Bacillus stearothermophilus.

24. The composition of any of paragraphs 22-23, wherein the alpha-amylase is from a strain of Bacillus stearothermophilus, in particular a variant of a Bacillus stearothermophilus alpha-amylase, such as the one shown in SEQ ID NO: 6.

25. The composition of any of paragraphs 23-24, wherein the Bacillus stearothermophilus alpha-amylase or variant thereof is truncated, preferably to have around 491 amino acids, such as from 480-495 amino acids.

26. The composition of any of paragraphs 23-25, wherein the Bacillus stearothermophilus alpha-amylase has a deletion at two positions within the range from positions 179 to 182, such as positions I181+G182, R179+G180, G180+I181, R179+I181, or G180+G182, preferably I181+G182, and optionally a N193F substitution, (using SEQ ID NO: 6 for numbering).

27. The composition of any of paragraphs 23-26, wherein the Bacillus stearothermophilus alpha-amylase has a substitution at position S242, preferably S242Q substitution.

28. The composition of any of paragraphs 23-27, wherein the Bacillus stearothermophilus alpha-amylase has a substitution at position E188, preferably E188P substitution.

29. The composition of any of paragraphs 21-28, wherein the alpha-amylase is selected from the group of Bacillus stearothermphilus alpha-amylase variants with the following mutations:

$$I {181}^{*} + G {182}^{*} + N 193 F + E 129 V + K 177 L + R 179 E ;$$

$$I {181}^{*} + G {182}^{*} + N 193 F + V 59 A + Q 89 R + E 129 V + K 177 L + R 179 E + H 208 Y + K 220 P + N 224 L + Q 254 S ;$$

$$I {181}^{*} + G {182}^{*} + N 193 F + V 59 A Q 89 R + E 129 V + K 177 L + R 179 S + Q 254 S + M 284 V ; and$$

$$I {181}^{*} + G {182}^{*} + N 193 F + E 129 V + K 177 L + R 179 E + K 220 P + N 224 L + S 242 Q + Q 254 S ( using SEQ ID NO : 6 for numbering ) .$$

30. The composition of any of paragraphs 21-29, wherein the alpha-amylase variant has at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 6.

31. The composition of any of paragraphs 21-30, comprising a protease, particularly a serine protease or a metallo-protease, such as an S8 serine protease.

32. The composition of paragraph 31, comprising a protease, preferably a protease selected from a Pyrococcus sp protease, e.g. a Pyrococcus furiosus protease shown as SEQ ID NO: 8, or a protease having at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 8.

33. The composition of paragraph 31, comprising a protease, preferably a protease selected from a Thermoascus sp protease, e.g., a Thermoascus aurantiacus protease, particularly a variant of a Thermoascus aurantiacus protease, SEQ ID NO: 7, comprising one of the specific combinations of substitutions:
D79L+S87P+A112P+D142L;
D79L+S87P+D142L; or
A27K+D79L+Y82F+S87G+D104P+A112P+A126V+D142L, and wherein the protease has at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 7.

34. The composition of paragraph 31, comprising a protease, preferably a protease selected from a Palaeococcus sp. S8 protease, particularly a Palaeococcus ferrophilus S8 protease shown as SEQ ID NO: 9 or a protease having at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 9.

35. The composition of any of paragraphs 21-34, comprising a thermo-stable glucoamylase, preferably a Penicillium oxalicum glucoamylase, more preferably a variant of a Penicillium oxalicum glucoamylase shown as SEQ ID NO: 16, comprising a K79V substitution (using SEQ ID NO: 16 for numbering), and further comprising one of the following mutations:

$$P 11 F + T 65 A + Q 327 F ; or$$

$$P 2 N + P 4 S + P 11 F + T 65 A + Q 327 F ; or$$

$$P 11 F + D 26 C + K33C + T 65 A + Q 327 F ; or$$

$$P 2 N + P 4 S + P 11 F + T 65 A + Q 327 F + E 501 V + Y 504 T ; or$$

$$P 2 N + P 4 S + P 11 F + T 65 A + Q 327 F + E 501 V + Y 504 T ; or$$

$$P 11 F + T 65 A + Q 327 W + E 501 V + Y 504 T ,$$

and wherein, the glucoamylase has at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the polypeptide of SEQ ID NO: 16.

36. A method of producing a variant pullulanase according to any of the paragraphs 1-17, comprising cultivating the host cell of paragraph 20 under conditions conducive for production of the polypeptide.

37. A process for producing a syrup from starch-containing material comprising the steps of:
a) liquefying the starch-containing material at a temperature above the initial gelatinization temperature using an alpha-amylase and a variant pullulanase of any of the paragraphs 1-17;
b) saccharifying using a glucoamylase.

38. A process for producing fermentation products from starch-containing material comprising the steps of:
a) liquefying the starch-containing material at a temperature above the initial gelatinization temperature using an alpha-amylase and a variant pullulanase of any of the paragraphs 1-17;
b) saccharifying using a glucoamylase;
c) fermenting using a fermenting organism.

39. The process of any of paragraphs 37-38, wherein the glucoamylase present and/or added in saccharification step b) and/or fermentation step c) is of fungal origin, preferably from a stain of Aspergillus, preferably A. niger, A. awamori, or A. oryzae; or a strain of Trichoderma, preferably T. reesei; or a strain of Talaromyces, preferably T. emersonii, or a strain of Trametes, preferably T. cingulata, or a strain of Pycnoporus, or a strain of Gloeophyllum, such as G. sepiarium or G. trabeum, or a strain of Nigrofomes.

40. The process of paragraph 39, wherein the glucoamylase is derived from Talaromyces emersonii, such as the one shown in SEQ ID NO: 10 herein.

41. The process of paragraph 40, wherein the glucoamylase is selected from the group consisting of:
(i) a glucoamylase comprising the polypeptide of SEQ ID NO: 10 herein;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 10.

42. The process of paragraph 39, wherein the glucoamylase is derived from Gloeophyllum sepiarium, such as the one shown in SEQ ID NO: 12.

43. The process of paragraph 42, wherein the glucoamylase is selected from the group consisting of:
(i) a glucoamylase comprising the polypeptide of SEQ ID NO: 12;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 12.

44. The process of paragraph 39, wherein the glucoamylase is derived from Gloeophyllum trabeum such as the one shown in SEQ ID NO: 13.

45. The process of claim 44, wherein the glucoamylase is selected from the group consisting of:
(i) a glucoamylase comprising the polypeptide of SEQ ID NO: 13;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 13.

46. The process of any of paragraphs 39-45, wherein a glucoamylase in combination with an alpha-amylase are present in saccharification and/or fermentation.

47. The process of paragraph 46, wherein the alpha-amylase present in saccharification and/or fermentation is of fungal or bacterial origin.

48. The process of paragraphs 46-47, wherein the alpha-amylase present and/or added in saccharification and/or fermentation is derived from a strain of the genus Rhizomucor, preferably a strain the Rhizomucor pusillus, such as a Rhizomucor pusillus alpha-amylase hybrid having an Aspergillus niger linker and starch-binding domain, such as the one included in SEQ ID NO: 15.

49. The process of any of paragraphs 46-48, wherein the alpha-amylase present in saccharification and/or fermentation is selected from the group consisting of:
(i) an alpha-amylase comprising the polypeptide of SEQ ID NO: 15;
(ii) an alpha-amylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 15.

50. The process of paragraph 49, wherein the alpha-amylase comprises one or more of the following substitutions: G128D, D143N, preferably G128D+D143N, using SEQ ID NO: 15 for numbering.

51. The process of any of paragraphs 39-50, further comprising, prior to the liquefaction step a), the steps of:
i) reducing the particle size of the starch-containing material, preferably by dry milling;
ii) forming a slurry comprising the starch-containing material and water.

52. The process of any of paragraphs 37-51, wherein at least 50%, preferably at least 70%, more preferably at least 80%, especially at least 90% of the starch-containing material fit through a sieve with #6 screen.

53. The process of any of paragraphs 37-52, wherein the pH in liquefaction is between above 4.5-6.5, such as around 4.8, or a pH between 5.0-6.2, such as 5.0-6.0, such as between 5.0-5.5, such as around 5.2, such as around 5.4, such as around 5.6, such as around 5.8.

54. The process of any of paragraphs 37-53, wherein the temperature in liquefaction is above the initial gelatinization temperature, such as in the range from 70-100° C., such as between 75-95° C., such as between 75-90° C., preferably between 80-90° C., especially around 85° C.

55. The process of any of paragraphs 37-54, wherein a jet-cooking step is carried out before liquefaction in step a).

56. The process of paragraph 55, wherein the jet-cooking is carried out at a temperature between 110-145° C., preferably 120-140° C., such as 125-135° C., preferably around 130° C. for about 1-15 minutes, preferably for about 3-10 minutes, especially around about 5 minutes.

57. The process of any of paragraphs 37-56, wherein saccharification is carried out at a temperature from 20-75° C., preferably from 40-70° C., such as around 60° C., and at a pH between 4 and 5.

58. The process of any of paragraphs 38-57, wherein fermentation or simultaneous saccharification and fermentation (SSF) is carried out at a temperature from 25° C. to 40° C., such as from 28° C. to 35° C., such as from 30° C. to 34° C., preferably around about 32° C.

59. The process of any of paragraphs 38-58, wherein the fermentation product is recovered after fermentation, such as by distillation.

60. The process of any of paragraphs 38-59, wherein the fermentation product is an alcohol, preferably ethanol, especially fuel ethanol, potable ethanol and/or industrial ethanol.

61. The process of any of paragraphs 37-60, wherein the starch-containing starting material is whole grains.

62. The process of any of paragraphs 37-61, wherein the starch-containing material is derived from corn, wheat, barley, rye, milo, sago, cassava, manioc, tapioca, sorghum, rice or potatoes.

63. The process of any of paragraphs 37-62, wherein a composition of any of claims 21-35 is added/is present in liquefaction.

64. The process of any of paragraphs 38-63, wherein the fermenting organism is yeast, preferably a strain of Saccharomyces, especially a strain of Saccharomyces cerevisiae.

65. The recombinant host cell according to paragraph 20, wherein the host cell is a yeast host cell, particularly a strain of Saccharomyces, more particularly Saccharomyces cerevisiae.

66. A use of the host cell according to paragraph 20 or 65, in fermentation of hydrolysed starch.

67. A use of the variant pullulanase of any of the paragraphs 1-17 in a brewing process.

68. A use of the variant pullulanase of any of the paragraphs 1-17 in liquefaction of starch-containing material.

69. A method of producing a brewer's wort comprising adding to a mash, a pullulanase of any of the paragraphs 1-17.

The present invention is further described by the following examples that should not be construed as limiting the scope of the invention.

### EXAMPLES

### Enzymes

Protease PfuS: Protease derived from Pyrococcus furiosus shown in SEQ ID NO: 8.

Alpha-Amylase BE369 (AA369): Bacillus stearothermophilus alpha-amylase disclosed herein as SEQ ID NO: 5, and further having the mutations: 1181*+G182*+N193F+V59A+Q89R+E129V+K177L+R179E+Q254S+M284V truncated to 491 amino acids (using SEQ ID NO: 6 for numbering).

Ms-trehalase: Myceliophthora sepedonium trehalase (SEQ ID NO: 30 in WO2016/205127) and SEQ ID NO: 17 herein.

Alpha-amylase blend AA: Blend comprising Alpha-amylase AA369, and protease PfuS (dosing: 2.1 μg EP/g DS AA369, 3.0 μg EP/g DS PfuS, where EP is enzyme protein and DS is total dry solids).

Glucoamylase A blend: Blend comprising Talaromyces emersonii glucoamylase (Te AMG) disclosed as SEQ ID NO: 34 in WO99/28448, Trametes cingulata glucoamylase (Tc AMG) disclosed as SEQ ID NO: 2 in WO 06/69289, and Rhizomucor pusillus alpha-amylase with Aspergillus niger glucoamylase linker and starch binding domain (SBD) (Rp AA) disclosed in SEQ ID NO: 15 herein having the following substitutions G128D+D143N using SEQ ID NO: 15 for numbering (activity ratio in AGU:AGU:FAU-F is about 29:8:1).

Glucoamylase B blend: Same as glucoamylase blend A further having a cellulase composition containing a Trichoderma reesei cellulase preparation containing Aspergillus fumigatus cellobiohydrolase I (WO 2011/057140), Aspergillus fumigatus cellobiohydrolase II (WO 2011/057140), Aspergillus fumigatus beta-glucosidase variant (WO 2012/044915), and Penicillium sp. (emersonii) GH61 polypeptide (WO 2011/041397), and Myceliophthora sepedonium trehalase (SEQ ID NO: 30 in WO2016/205127) and SEQ ID NO: 17 herein (dosing: Te AMG 60 μg EP/gDS; Tc AMG 20 μg EP/gDS; Rp AA 11 μg EP/gDS; Cellulase composition 30 μg EP/gDS, Ms trehalase 1 μg EP/gDS).

Yeast: ETHANOL RED™ from Fermentis, USA

### Assays

### Protease Assays

### 1) Kinetic Suc-AAPF-pNA Assay

pNA substrate: Suc-AAPF-pNA (Bachem L-1400).
Temperature: Room temperature (25° C.)
Assay buffers: 100 mM succinic acid, 100 mM HEPES, 100 mM CHES, 100 mM CABS,
   1 mM CaCl₂, 150 mM KCl, 0.01% Triton X-100 adjusted to pH-values 2.0, 3.0, 4.0, 5.0, 6.0, 7.0, 8.0, 9.0, 10.0, and 11.0 with HCl or NaOH.

20 μl protease (diluted in 0.01% Triton X-100) was mixed with 100 μl assay buffer. The assay was started by adding 100 μl pNA substrate (50 mg dissolved in 1.0 ml DMSO and further diluted 45× with 0.01% Triton X-100). The increase in OD₄₀₅ was monitored as a measure of the protease activity.

### 2) Endpoint Suc-AAPF-pNA AK Assay

pNA substrate: Suc-AAPF-pNA (Bachem L-1400).
Temperature: controlled (assay temperature).
Assay buffer: 100 mM succinic acid, 100 mM HEPES, 100 mM CHES, 100 mM CABS,
   1 mM CaCl₂, 150 mM KCl, 0.01% Triton X-100, pH 7.0.

200 μl pNA substrate (50 mg dissolved in 1.0 ml DMSO and further diluted 45× with the Assay buffer) were pipetted in an Eppendorf tube and placed on ice. 20 μl protease sample (diluted in 0.01% Triton X-100) was added. The assay was initiated by transferring the Eppendorf tube to an Eppendorf thermomixer, which was set to the assay temperature. The tube was incubated for 15 minutes on the Eppendorf thermomixer at its highest shaking rate (1400 rpm.). The incubation was stopped by transferring the tube back to the ice bath and adding 600 μl 500 mM Succinic acid/NaOH, pH 3.5. After mixing the Eppendorf tube by vortexing 200 μl mixture was transferred to a microtiter plate. OD₄₀₅ was read as a measure of protease activity. A buffer blind was included in the assay (instead of enzyme).

### Glucoamylase Activity (AGU)

Glucoamylase activity may be measured in Glucoamylase Units (AGU).

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

AMG incubation:

| | | |
|---|---|---|
| Substrate: | maltose 23.2 | mM |
| Buffer: | acetate 0.1 | M |
| pH: | 4.30 | ± 0.05 |
| Incubation | 37° C. | ± 1 |
| temperature: | | |
| Reaction time: | 5 | minutes |
| Enzyme working | 0.5-4.0 | AGU/mL |
| range: | | |

Color reaction:

| GlucDH: | 430 | U/L |
|---|---|---|
| Mutarotase: | 9 | U/L |
| NAD: | 0.21 | mM |
| Buffer: | phosphate 0.12 M; | |
| | 0.15 M NaCl | |
| pH: | 7.60 | ± 0.05 |
| Incubation | 37° C. | ± 1 |
| ternperature: | | |
| Reaction time: | 5 | minutes |
| Wavelength: | 340 | nm |

A folder (EB-SM-0131.02/01) describing this analytical method in more detail is available on request from Novozymes A/S, Denmark, which folder is hereby included by reference.

### Alpha-Amylase Activity Assays

### Acid Alpha-Amylase Activity (AFAU)

Acid alpha-amylase activity may be measured in AFAU (Acid Fungal Alpha-amylase Units), which are determined relative to an enzyme standard. 1 AFAU is defined as the amount of enzyme which degrades 5.260 mg starch dry matter per hour under the below mentioned standard conditions.

Acid alpha-amylase, an endo-alpha-amylase (1,4-alpha-D-glucan-glucanohydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

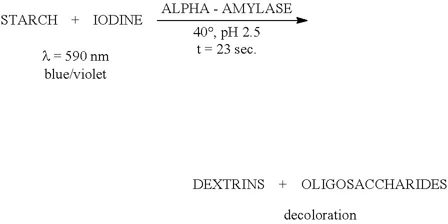

### Standard Conditions/Reaction Conditions

Substrate: Soluble starch, approx. 0.17 g/L
Buffer: Citrate, approx. 0.03 M
Iodine (I2): 0.03 g/L
CaCl₂: 1.85 mM
pH: 2.50±0.05
Incubation 40° C.
temperature:
Reaction time: 23 seconds
Wavelength: 590 nm
Enzyme 0.025 AFAU/mL
concentration:
Enzyme working 0.01-0.04 AFAU/mL
range:

A folder EB-SM-0259.02/01 describing this analytical method in more detail is available upon request to Novozymes A/S, Denmark, which folder is hereby included by reference.

### Determination of FAU-F

FAU-F Fungal Alpha-Amylase Units (Fungamyl) is measured relative to an enzyme standard of a declared strength.

Reaction conditions

| Temperature | 37° | C. |
|---|---|---|
| pH | 7.15 | |
| Wavelength | 405 | nm |
| Reaction time | 5 | min |
| Measuring time | 2 | min |

A folder (EB-SM-0216.02) describing this standard method in more detail is available on request from Novozymes A/S, Denmark, which folder is hereby included by reference.

### Alpha-Amylase Activity (KNU)

The alpha-amylase activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C.+/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

### pNP-G7 Assay

The alpha-amylase activity may be determined by a method employing the G7-pNP substrate. G7-pNP which is an abbreviation for 4,6-ethylidene(G₇)-p-nitrophenyl(G₁)-┘,D-maltoheptaoside, a blocked oligosaccharide which can be cleaved by an endo-amylase, such as an alpha-amylase. Following the cleavage, the alpha-Glucosidase included in the kit digest the hydrolysed substrate further to liberate a free PNP molecule which has a yellow color and thus can be measured by visible spectophometry at lambda=405 nm (400-420 nm.). Kits containing G7-pNP substrate and alpha-Glucosidase is manufactured by Roche/Hitachi (cat. No. 11876473).

### Reagents

The G7-pNP substrate from this kit contains 22 mM 4,6-ethylidene-G7-pNP and 52.4 mM HEPES (2-[4-(2-hydroxyethyl)-1-piperazinyl]-ethanesulfonic acid), pH 7.0).

The alpha-Glucosidase reagent contains 52.4 mM HEPES, 87 mM NaCl, 12.6 mM MgCl₂, 0.075 mM CaCl₂, ≥4 kU/L alpha-glucosidase).

The substrate working solution is made by mixing 1 mL of the alpha-Glucosidase reagent with 0.2 mL of the G7-pNP substrate. This substrate working solution is made immediately before use.

Dilution buffer: 50 mM MOPS, 0.05% (w/v) Triton X100 (polyethylene glycol p-(1,1,3,3-tetramethylbutyl)-phenyl ether (C₁₄H₂₂O(C₂H₄O), (n=9-10))), 1 mM CaCl2, pH8.0.

### Procedure

The amylase sample to be analyzed is diluted in dilution buffer to ensure the pH in the diluted sample is 7. The assay is performed by transferring 20 μl diluted enzyme samples to 96 well microtiter plate and adding 80 μl substrate working solution. The solution is mixed and pre-incubated 1 minute at room temperature and absorption is measured every 20 sec. over 5 minutes at OD 405 nm.

The slope (absorbance per minute) of the time dependent absorption-curve is directly proportional to the specific activity (activity per mg enzyme) of the alpha-amylase in question under the given set of conditions. The amylase sample should be diluted to a level where the slope is below 0.4 absorbance units per minute.

### EnzChek® Assay

For the determination of residual amylase activity an EnzChek® Ultra Amylase Assay Kit (E33651, Invitrogen, La Jolla, CA, USA) may be used.

The substrate is a corn starch derivative, DQ™ starch, which is corn starch labeled with BODIPY® FL dye to such a degree that fluorescence is quenched. One vial containing approx. 1 mg lyophilized substrate is dissolved in 100 microliters of 50 mM sodium acetate (pH 4.0). The vial is vortexed for 20 seconds and left at room temperature, in the dark, with occasional mixing until dissolved. Then 900 microliters of 100 mM acetate, 0.01% (w/v) TRITON® X100, 0.125 mM CaCl₂, pH 5.5 is added, vortexed thoroughly and stored at room temperature, in the dark until ready to use. The stock substrate working solution is prepared by diluting 10-fold in residual activity buffer (100 mM acetate, 0.01% (w/v) TRITON® X100, 0.125 mM CaCl₂, pH 5.5). Immediately after incubation the enzyme is diluted to a concentration of 10-20 ng enzyme protein/ml in 100 mM acetate, 0.01% (W/v) TRITON® X100, 0.125 mM CaCl₂, pH 5.5.

For the assay, 25 microliters of the substrate working solution is mixed for 10 second with 25 microliters of the diluted enzyme in a black 384 well microtiter plate. The fluorescence intensity is measured (excitation: 485 nm, emission: 555 nm) once every minute for 15 minutes in each well at 25° C. and the Vₘₐₓ is calculated as the slope of the plot of fluorescence intensity against time. The plot should be linear and the residual activity assay has been adjusted so that the diluted reference enzyme solution is within the linear range of the activity assay.

### Pullulanase Assays

### Pullulanase Activity (NPUN) Assay

Endo-pullulanase activity in NPUN is measured relative to a Novozymes pullulanase standard. One pullulanase unit (NPUN) is defined as the amount of enzyme that releases 1 micro mol glucose per minute under the standard conditions (0.7% red pullulan (Megazyme), pH 5, 40° C., 20 minutes). The activity is measured in NPUN/ml using red pullulan.

1 mL diluted sample or standard is incubated at 40° C. for 2 minutes. 0.5 mL 2% red pullulan, 0.5 M KCl, 50 mM citric acid, pH 5 are added and mixed. The tubes are incubated at 40° C. for 20 minutes and stopped by adding 2.5 ml 80% ethanol. The tubes are left standing at room temperature for 10-60 minutes followed by centrifugation 10 minutes at 4000 rpm. OD of the supernatants is then measured at 510 nm and the activity calculated using a standard curve.

### Phadebas Thermostability Assay

The culture supernatants containing desired enzymes was mixed with same volume of pH 5.0 400 mM NaOAc buffer. Twenty microliter of this mixture was dispensed into either 96-well plate or 8-strip PCR tube, and then heated by thermal cycler at various temperatures for 30 min. Those samples were mixed with 100 μl of substrate solution containing 2% (w/v) phadebas [magle life science] in pH 5.0 200 mM NaOAc buffer, and incubated at 70° C. for 30 min for enzymatic reaction. After the reaction, 50 μl of 18% Acetic acid was added to stop the reaction. Eighty microliter of reaction supernatant was taken out and its OD₆₀₀ value was read by photometer to evaluate the enzyme activity.

### Phadebas Thermoprofile Assay

The culture supernatants containing desired enzymes was mixed with same volume of pH 5.0 400 mM NaOAc buffer. Twenty microliter of this mixture was dispensed into 96-well plate or 8-strip PCR tubes, and then mixed with 100 μl of substrate solution containing 2% (w/v) phadebas [magle life science] in pH 5.0 200 mM NaOAc buffer. Those samples were incubated at various temperatures for 30 min for enzymatic reaction. After the reaction, 50 μl of 18% Acetic acid was added to stop the reaction. Eighty microliter of reaction supernatant was taken out and its OD₆₀₀ value was read by photometer to evaluate the enzyme activity.

### Example 1: Construction of Pullulanase Libraries

Pullulanase libraries were constructed as follows.

A forward or reverse primer having NNK or desired mutation(s) at target site(s) with 15 bp overlaps each other were designed. Inverse PCR, which means amplification of entire plasmid DNA sequences by inversely directed primers, were carried out with appropriate template plasmid DNA (e.g. plasmid DNA containing P604 encoding gene) by the following conditions. The resultant PCR fragments were purified by QIAquick Gel extraction kit [QIAGEN], and then introduced into Escherichia coli DH5α Competent HIGH [TOYOBO]. The plasmid DNAs were extracted from E. coli transformants by MagExtractor plasmid extraction kit [TOYOBO], and then introduced into B. subtilis competent cells.

### PCR Reaction Mix

PrimeSTAR Max DNA polymerase [TaKaRa]
Total 25 μl
1.0 μl Template DNA (1 ng/μl)
9.5 μl H₂O
12.5 μl 2× PrimeSTAR Max pre-mix
1.0 μl Forward primer (5 μM)
1.0 μl Reverse primer (5 μM)

### PCR Program

98° C./2 min
25× (98° C./10 sec, 60° C./15 sec, 72° C./2 min)
10° C./hold

### Example 2: Screening for Better Thermostability

B. subtilis libraries constructed as in EXAMPLE 1 were fermented in either 96-well or 24-well MTP containing 10R-av-30C medium (6.0 g/L (NH₄)₂HPO₄, 26 g/L Bacto pepton, 1.2 g/L MgSO₄.7H₂O, 12 g/L KH₂PO₄, 5.0 g/L Na₂HPO₄, 1.8 g/L K₂SO₄, 0.1 g/L CaCl₂.2H₂O, 33 g/L glucose, 4.9 mg/L MnSO₄.5H₂O, 19.7 mg/L FeSO₄.7H₂O, 1.0 mg/L CuSO₄.5H₂O, 3.0 mg/L ZnCl₂, 196 mg/L Citric acid) with 8.0 mg/L chloramphenicol at 800 rpm, 37° C., overnight. Then, pullulanase activities in culture supernatants were measured at several temperatures by Phadebas assay described as follows.

### Phadebas Thermostability Assay

The culture supernatants containing desired enzymes was mixed with same volume of pH 5.0 400 mM NaOAc buffer. Twenty microliter of this mixture was dispensed into either 96-well plate or 8-strip PCR tube, and then heated by thermal cycler at various temperatures for 30 min. Those samples were mixed with 100 μl of substrate solution containing 2% (w/v) phadebas [magle life science] in pH 5.0 200 mM NaOAc buffer, and incubated at 70° C. for 30 min for enzymatic reaction. After the reaction, 50 μl of 18% Acetic acid was added to stop the reaction. Eighty microliter of reaction supernatant was taken out and its OD₆₀₀ value was read by photometer to evaluate the enzyme activity.

### Phadebas Thermoprofile Assay

The culture supernatants containing desired enzymes was mixed with same volume of pH 5.0 400 mM NaOAc buffer. Twenty microliter of this mixture was dispensed into 96-well plate or 8-strip PCR tubes, and then mixed with 100 μl of substrate solution containing 2% (w/v) phadebas [magle life science] in pH 5.0 200 mM NaOAc buffer. Those samples were incubated at various temperatures for 30 min for enzymatic reaction. After the reaction, 50 μl of 18% Acetic acid was added to stop the reaction. Eighty microliter of reaction supernatant was taken out and its OD₆₀₀ value was read by photometer to evaluate the enzyme activity.

### PAHBAH-maltodextrin (DE3) Assay

### Substrate Solution

1 g maltodextrin (pindex 100 from MATSUTANI chemical industry Co., Ltd.)
5 ml 50 mM sodium acetate buffer, pH 5

### PAHBAH Solution

0.0552 g Bismuth (III)-acetate
0.2 g PAHBAH
0.5 g Potassium sodium tartrate, tetrahydrate
10 ml 500 mM NaOH

Ten ul of enzyme samples were mixed with 110 ul of substrate solution and incubated at set temperatures for 2 hours (85° C.) or 30 min (91° C.). Ten ul of 0.5 N NaOH was added to stop reaction and cooled the tubes to 55° C. The reaction mixtures were diluted 40-fold with 50 mM sodium acetate buffer, pH 5. Forty ul of PAHBAH solution was added to 120 ul of diluted mixtures, incubated for another 20 min at 55° C. and the absorbance at A405 was read.

TABLE 1a
List of the relative activity of pullulanase variants when
compared with their parents (P604 or P609)

| | <Thermostability> | <Thermoprofile> |
|---|---|---|
| | Relative activity | Relative activity |
| | of 81.5° C./80° C. (%) | of 81.5° C./80° C. (%) |
| P604 | 43% | 56% |
| P609 | 58% | 57% |
| | <Thermostability> | <Thermoprofile> |
| | Relative activity | Relative activity |
| | of 81° C./80° C. (%) | of 81.5° C./70° C. (%) |
| P609 | 50% | 39% |
| P624 | 52% | 34% |
| P625 | 55% | 49% |
| P629 | 69% | 39% |
| | <Thermostability> | <Thermoprofile> |
| | Relative activity | Relative activity |
| | of 81° C./80° C. (%) | of 81.5° C./80° C. (%) |
| P604 | 38% | 48% |
| P609 | 48% | 55% |
| P630 | 53% | 59% |
| P631 | 71% | 83% |
| P632 | 67% | 73% |
| | <Thermostability> | |
| | Relative activity of 81° C./70° C. (%) | |
| P604 | 66% | |
| P633 | 78% | |
| P634 | 75% | |
| P635 | 71% | |
| | <Thermostability> | |
| | Relative activity of 81° C./70° C. (%) | |
| P609 | 60% | |
| P638 | 68% | |
| P641 | 61% | |
| P643 | 54% | |
| P644 | 66% | |
| | <Thermostability> | <Thermoprofile> |
| | Relative activity | Relative activity |
| | of 83° C./70° C. (%) | of 81.5° C./80° C. (%) |
| P609 | 10% | 32% |
| P639 | 46% | 59% |
| P640 | 24% | 44% |
| | <Thermostability> | <Thermoprofile> |
| | Relative activity | Relative activity |
| | of 81° C./80° C. (%) | of 81.5° C./80° C. (%) |
| P609 | 28% | 24% |
| P645 | 42% | 15% |
| P646 | 35% | 21% |

TABLE 1b
List of the relative activity of pullulanase
variants when compared with their parents (P632)

| | <Thermostability> |
|---|---|
| | Relative activity of 83° C./70° C. (%) |
| P632 | 12% |
| P648 | 19% |
| P649 | 19% |
| P650 | 24% |
| P651 | 28% |
| P652 | 24% |
| P653 | 26% |
| P654 | 20% |
| P655 | 16% |
| P656 | 46% |
| P657 | 26% |

TABLE 1c
List of the relative activity of pullulanase
variants when compared with their parents (P656)

| | <Thermostability> |
|---|---|
| | Relative activity of 83° C./70° C. (%) |
| P656 | 53% |
| P675 | 61% |
| P676 | 57% |
| P677 | 58% |
| P678 | 62% |
| P679 | 63% |
| P680 | 63% |
| P681 | 62% |
| P682 | 64% |
| P656 | 15% |
| P683 | 25% |
| P684 | 30% |
| P685 | 38% |
| P698 | 43% |

TABLE 1d
List of the relative activity of pullulanase variants when
compared with their parents (P685 or P698)

| | <Thermostability> |
|---|---|
| | Relative activity of 84° C./70° C. (%) |
| P685 | 19% |
| P698 | 21% |
| P699 | 24% |
| P700 | 15% |
| P685 | 11% |
| P698 | 14% |
| P703 | 13% |
| P725 | 16% |
| | <Thermostability> |
| | Relative activity of 83° C./70° C. (%) |
| P685 | 32% |
| P698 | 38% |
| P718 | 13% |
| P719 | 30% |

TABLE 1e
List of the relative activity of pullulanase variants
when compared with their parents (P719)

| | <Thermostability> | <Thermoprofile> |
|---|---|---|
| | Relative activity | Relative activity |
| | of 83° C./80° C. (%) | of 83° C./80° C. (%) |
| P719 | 43% | 34% |
| P731 | 44% | 46% |
| P741 | 64% | 36% |
| | <Thermostability> | |
| | Relative activity of 83° C./70° C. (%) | |
| P719 | 9% | |
| P753 | 12% | |
| P765 | 22% | |
| P766 | 28% | |
| | <Thermostability> | <Thermoprofile> |
| | Relative activity of | Relative activity of |
| | 84° C./80° C. (%) | 85° C./80° C. (%) |
| P719 | not tested | 15% |
| P753 | 7% | 22% |
| P765 | 15% | 41% |
| P766 | 16% | 23% |
| P778 | 12% | 30% |
| P779 | 24% | 35% |
| P781 | 24% | 31% |
| P782 | 18% | 36% |
| P783 | 22% | 33% |

TABLE 1f
List of the relative activity of pullulanase variants
when compared with their parents (P781)

| | <Thermostability> | <Thermoprofile> |
|---|---|---|
| | Relative activity | Relative activity |
| | of 85° C./80° C. (%) | of 85° C./83° C. (%) |
| P781 | 4% | 49% |
| P787 | 7% | 50% |
| P789 | 5% | 40% |
| P791 | 3% | 47% |
| P795 | 17% | 49% |
| P797 | 14% | 54% |

TABLE 1g
List of the relative activity of pullulanase
variants when compared with their parents (P797)

| | <Thermostability> | |
|---|---|---|
| | Relative activity of 84° C./80° C. (%) | |
| P797 | 12% | |
| P823 | 36% | |
| P845 | 22% | |
| P846 | 17% | |
| P847 | 37% | |
| P848 | 13% | |
| P855 | 16% | |
| P856 | 32% | |
| P861 | 16% | |
| | <Thermostability> | <Thermoprofile> |
| | Relative activity of | Relative activity of |
| | 84° C./80° C. (%) | 85° C./84° C. (%) |
| P797 | 15% | 39% |
| P865 | 14% | 40% |
| P866 | 11% | 45% |
| P876 | 17% | 52% |
| P881 | 20% | 37% |
| P882 | 6% | 45% |
| P883 | 7% | 55% |
| P893 | 25% | 48% |
| | <Thermostability> | |
| | Relative activity of 84° C./80° C. (%) | |
| P797 | 3% | |
| P823 | 15% | |
| P897 | 13% | |
| P898 | 17% | |
| P899 | 31% | |
| P900 | 42% | |
| P901 | 40% | |
| P902 | 45% | |
| P903 | 42% | |
| P905 | 31% | |
| P906 | 33% | |
| P907 | 45% | |
| P940 | 34% | |
| P941 | 41% | |
| | <Thermostability> | <Thermoprofile> |
| | Relative activity of | Relative activity of |
| | 84° C./80° C. (%) | 84° C./80° C. (%) |
| P797 | 20% | 33% |
| P922 | 22% | 28% |
| P931 | 26% | 37% |
| P933 | 25% | 30% |
| | <Thermostability> | <Thermoprofile> |
| | Relative activity of | Relative activity of |
| | 86° C./85° C. (%) | 85° C./80° C. (%) |
| P797 | 2% | 53% |
| P823 | 9% | 51% |
| P901 | 18% | 55% |
| P944 | 63% | 64% |
| | <Thermostability> | <Thermoprofile> |
| | Relative activity of | Relative activity of |
| | 84° C./80° C. (%) | 85° C./84° C. (%) |
| P797 | 11% | 49% |
| P995 | 9% | 52% |
| P1025 | 8% | 58% |
| P1026 | 19% | 54% |

TABLE 1h
List of the relative activity of pullulanase variants when
compared with their parents (P944 or P993)

| | <Thermostability> | <Thermoprofile> |
|---|---|---|
| | Relative activity of | Relative activity of |
| | 87° C./86° C. (%) | 86° C./80° C. (%) |
| P944 | 17% | 15% |
| P990 | 24% | 18% |
| P991 | 24% | 16% |
| P992 | 28% | 19% |
| P993 | 25% | 16% |
| | <Thermostability> | <Thermoprofile> |
| | Relative activity | Relative activity |
| | of 85° C./80° C. (%) | of 86° C./85° C. (%) |
| P993 | 66% | 45% |
| P1031 | 74% | 38% |
| P1033 | 79% | 53% |
| P1034 | 65% | 40% |
| | <Thermostability> | <Thermoprofile> |
| | Relative activity | Relative activity |
| | of 87° C./85° C. (%) | of 86° C./80° C. (%) |
| P993 | 49% | 73% |
| P1033 | 59% | 86% |
| P1037 | 71% | 95% |

TABLE 2
Substitutions of thermostabilized variants on P604

| | |
|---|---|
| JPUL609 | K370S |
| JPUL624 | Q279R K370S |
| JPUL625 | H321E K370S |
| JPUL629 | K370S V821* S822* P823* D824* H825* G826* K827* K828* |
| JPUL630 | K370S Q399N |
| JPUL631 | K370S N401D |
| JPUL632 | K370S F432V |
| JPUL633 | V196T K370S |
| JPUL634 | V196C K370S |
| JPUL635 | T197I K370S |
| JPUL638 | K370S V460E |
| JPUL639 | K370S T486A |
| JPUL640 | K370S T486V |
| JPUL641 | K370S I490L |
| JPUL643 | K370S V514A |
| JPUL644 | K370S T529L |
| JPUL645 | K370S S531R |
| JPUL646 | K370S Q595R |
| JPUL648 | H321E K370S F432V |
| JPUL649 | K370S F432V V821* S822* P823* D824* H825* G826* K827* K828* |
| JPUL650 | K370S Q399N F432V |
| JPUL651 | K370S N401D F432V |
| JPUL652 | V196T K370S F432V |
| JPUL653 | V196C K370S F432V |
| JPUL654 | T197I K370S F432V |
| JPUL655 | K370S F432V G459E |
| JPUL656 | K370S F432V T486A |
| JPUL657 | K370S F432V T486V |
| JPUL675 | V196T K370S F432V T486A |
| JPUL676 | T197I K370S F432V T486A |
| JPUL677 | V196T T197I K370S F432V T486A |
| JPUL678 | K370S Q399N F432V T486A |
| JPUL679 | K370S N401D F432V T486A |
| JPUL680 | K370S Q399N N401D F432V T486A |
| JPUL681 | K370S S531R F432V T486A |
| JPUL682 | K370S Q595R F432V T486A |
| JPUL683 | K370S F432V T486A S531R Q595R |
| JPUL684 | V196T T197I K370S Q399N N401D F432V T486A |
| JPUL685 | V196T T197I K370S Q399N N401D F432V T486A Q595R |
| JPUL698 | T197I K370S N401D F432V T486A S531R Q595R |
| JPUL699 | T197I K370S N401D F432V T486A S531R Q595R V821* S822* P823* D824* |
| | H825* G826* K827* K828* |
| JPUL700 | T197I K370S Q399N N401D F432V T486A Q595R |
| JPUL703 | S103K T197I K370S N401D F432V T486A S531R Q595R |
| JPUL718 | V196T T197I K370S Q399N N401D F432V T486V Q595R |
| JPUL719 | T197I K370S N401D F432V T486V S531R Q595R |
| JPUL725 | V196T T197I K370S Q399N N401D F432V T486A S531R Q595R |
| JPUL731 | D77G T197I K370S N401D F432V T486V S531R Q595R |
| JPUL741 | T197I K370S N401D S402Q F432V T486V S531R Q595R |
| JPUL753 | T197I N283F K370S N401D F432V T486V S531R Q595R |
| JPUL765 | T197I N283F K370S N401D S402Q F432V T486V S531R Q595R |
| JPUL766 | T197I N283F K370S N401D F432V L435A T486V S531R Q595R |
| JPUL778 | D77G T197I N283F K370S N401D S402Q F432V T486V S531R Q595R |
| JPUL779 | T197I N283F K370S N401D S402Q F432V L435A T486V S531R Q595R |
| JPUL781 | D77G T197I N283F K370S N401D S402Q F432V L435A T486V S531R Q595R |
| JPUL782 | T197I N283F K370S N401D S402Q F432V L435A T486V S531R Q595R D649A |
| JPUL783 | D77G T197I N283F K370S N401D S402Q F432V L435A T486V S531R Q595R |
| | D649A |
| JPUL787 | D77G Q106W T197I N283F K370S N401D S402Q F432V L435A T486V S531R |
| | Q595R |
| JPUL789 | D77G A107D T197I N283F K370S N401D S402Q F432V L435A T486V S531R |
| | Q595R |
| JPUL791 | D77G V196C T197I N283F K370S N401D S402Q F432V L435A T486V S531R |
| | Q595R |
| JPUL795 | D77G T197I N283F K370S N401D S402Q F432V L435A T486V S531R N5830 |
| Q595R | |
| JPUL797 | D77G T197I N283F K370S N401D S402Q F432V L435A T486V S531R Q595R |
| | V821* S822* P823* D824* H825* G826* K827* K828* |
| JPUL823 | D77G T197I N283F K370S N401D S402Q F432V L435A T486V S531R Q595R |
| | D688A V821* S822* P823* D824* H825* G826* K827* K828* |
| JPUL845 | D77G T197I N283F D367G K370S N401D S402Q F432V L435A T486V S531R |
| | Q595R V821* S822* P823* D824* H825* G826* K827* K828* |
| JPUL846 | D77G T197I N283F D367N K370S N401D S402Q F432V L435A T486V S531R |
| | Q595R V821* S822* P823* D824* H825* G826* K827* K828* |
| JPUL847 | D77G T197I N283F K370S S375H N401D S402Q F432V L435A T486V S531R |
| | Q595R V821* S822* P823* D824* H825* G826* K827* K828* |
| JPUL848 | D77G T197I N283F K370S N382T N401D S402Q F432V L435A T486V S531R |
| | Q595R V821* S822* P823* D824* H825* G826* K827* K828* |
| JPUL855 | D77G T197I N283F K370S N401D S402Q N411L F432V L435A T486V S531R |
| | Q595R V821* S822* P823* D824* H825* G826* K827* K828* |
| JPUL856 | D77G T197I N283F K370S N401D S402Q Y412F F432V L435A T486V S531R |
| | Q595R V821* S822* P823* D824* H825* G826* K827* K828* |
| JPUL861 | D77G T197I N283F K370S N401D S402Q F432V Q434E L435A T486V S531R |
| | Q595R V821* S822* P823* D824* H825* G826* K827* K828* |
| JPUL865 | D77G T197I N283F K370S N401D S402Q F432V L435A R443G T486V S531R |
| | Q595R V821* S822* P823* D824* H825* G826* K827* K828* |
| JPUL866 | D77G T197I N283F K370S N401D S402Q F432V L435A I446V T486V S531R |
| | Q595R V821* S822* P823* D824* H825* G826* K827* K828* |
| JPUL876 | D77G T197I N283F K370S N401D 3402Q F432V L435A T486V Q498R S531R |
| | Q595R V821* S822* P823* D824* H825* G826* K827* K828* |
| JPUL881 | D77G T197I N283F K370S N401D S402Q F432V L435A T486V S531R A533I |
| | Q595R V821* S822* P823* D824* H825* G826* K827* K828* |
| JPUL882 | D77G T197I N283F K370S N401D S402Q F432V L435A T486V S531R N541D |
| | Q595R V821* S822* P823* D824* H825* G826* K827* K828* |
| JPUL883 | D77G T197I N283F K370S N401D S402Q F432V L435A T486V S531R A545I |
| | Q595R V821* S822* P823* D824* H825* G826* K827* K828* |
| JPUL893 | D77G T197I N283F K370S N401D S402Q F432V L435A T486V S531R L581F |
| | Q595R V821* S822* P823* D824* H825* G826* K827* K828* |
| JPUL897 | D77G T197I N283F K370S N401D S402Q F432V L435A H479N T486V S531R |
| | Q595R D688A V821* S822* P823* D824* H825* G826* K827* K828* |
| JPUL898 | D77G T197I N283F D367N K370S N401D S402Q F432V L435A T486V S531R |
| | Q595R D688A V821* S822* P823* D824* H825* G826* K827* K828* |
| JPUL899 | D77G T197I N283F K370S S375H N401D S402Q F432V L435A T486V S531R |
| | Q595R D688A V821* S822* P823* D824* H825* G826* K827* K828* |
| JPUL900 | D77G T197I N283F K370S N401D S402Q Y412F F432V L435A T486V S531R |
| | Q595R D688A V821* S822* P823* D824* H825* G826* K827* K828* |
| JPUL901 | D77G T197I N283F K370S N401D S402Q F432V L435A T486V S531R A533I |
| | Q595R D688A V821* S822* P823* D824* H825* G826* K827* K828* |
| JPUL902 | D77G T197I N283F K370S N401D S402Q F432V L435A T486V S531R L581F |
| | Q595R D688A V821* S822* P823* D824* H825* G826* K827* K828* |
| JPUL903 | D77G T197I N283F D367N K370S S375H N401D S402Q F432V L435A T486V |
| | S531R Q595R D688A V821* S822* P823* D824* H825* G826* K827* K828* |
| JPUL905 | D77G T197I N283F D367N K370S N401D S402Q Y412F F432V L435A T486V |
| | S531R Q595R D688A V821* S822* P823* D824* H825* G826* K827* K828* |
| JPUL906 | D77G T197I N283F D367N K370S N401D S402Q F432V L435A T486V S531R |
| | A533I Q595R D688A V821* S822* P823* D824* H825* G826* K827* K828* |
| JPUL907 | D77G T197I N283F D367N K370S N401D S402Q F432V L435A T486V S531R |
| | L581F Q595R D688A V821* S822* P823* D824* H825* G826* K827* K828* |
| JPUL922 | D77G T197I N283F K370S N401D S402Q F432V L435A T486V S531R Q595R |
| | V665I V821* S822* P823* D824* H825* G826* K827* K828* |
| JPUL931 | D77G T197I N283F K370S N401D S402Q F432V L435A T486V S531R Q595R |
| | F700L V821* S822* P823* D824* H825* G826* K827* K828* |
| JPUL933 | D77G T197I N283F K370S N401D S402Q F432V L435A T486V S531R Q595R |
| | P709I V821* S822* P823* D824* H825* G826* K827* K828* |
| JPUL940 | D77G T197I N283F D367N K370S S375H N401D S402Q Y412F F432V L435A |
| | T486V S531R Q595R D688A V821* S822* P823* D824* H825* G826* K827* |
| | K828* |
| JPUL941 | D77G T197I N283F D367N K370S S375H N401D S402Q F432V L435A T486V |
| | S531R A533I Q595R D688A V821* S822* P823* D824* H825* G826* K827* |
| | K828* |
| JPUL944 | D77G T197I N283F D367N K370S S375H N401D S402Q Y412F F432V L435A |
| | T486V S531R A533I L581F Q595R D688A V821* S822* P823* D824* H825* |
| | G826* K827* K828* |
| JPUL990 | D77G T197I N283F H321V D367N K370S S375H N401D S402Q Y412F F432V |
| | Q434E L435A T486V S531R A533I L581F Q595R D688A V821* S822* P823* |
| | D824* H825* G826* K827* K828* |
| JPUL991 | D77G T197I N283F H321V D367N K370S S375H N401D S402Q Y412F F432V |
| | L435A T486V Q498R S531R A533I L581F Q595R D688A V821* S822* P823* |
| | D824* H825* G826* K827* K828* |
| JPUL992 | D77G T197I N283F H321V D367N K370S S375H N401D S402Q Y412F F432V |
| | L435A T486V S531R A533I L581F Q595R D688A F700L V821* S822* P823* |
| | D824* H825* G826* K827* K828* |
| JPUL993 | D77G T197I N283F H321V D367N K370S S375H N401D S402Q Y412F F432V |
| | Q434E L435A T486V Q498R S531R A533I L581F Q595R D688A F700L V821* |
| | S822* P823* D824* H825* G826* K827* K828* |
| JPUL995 | F17Y D77G T197I N283F K370S N401D S402Q F432V L435A T486V S531R |
| | Q595R V821* S822* P823* D824* H825* G826* K827* K828* |
| JPUL1025 | D77G T197I N283F K370S N401D S402Q F432V L435A T486V S531R Q595R |
| | E804S V821* S822* P823* D824* H825* G826* K827* K828* |
| JPUL1026 | D77G T197I N283F K370S N401D S402Q F432V L435A T486V S531R Q595R |
| | G811R V821* S822* P823* D824* H825* G826* K827* K828* |
| JPUL1031 | D77G T197I N283F H321V K370S S375H N401D S402Q Y412F F432V Q434E |
| | L435A H479N T486V Q498R S531R A533I L581F Q595R D688A F700L V821* |
| | S822* P823* D824* H825* G826* K827* K828* |
| JPUL1033 | D77G A190I T197I N283F K370S S375H N401D S402Q Y412F F432V Q434E |
| | L435A H479N T486V Q498R S531R A533I L581F Q595R D688A F700L V821* |
| | S822* P823* D824* H825* G826* K827* K828* |
| JPUL1034 | D77G T197I T262V N283F K370S S375H N401D S402Q Y412F F432V Q434E |
| | L435A H479N T486V Q498R S531R A533I L581F Q595R D688A F700L V821* |
| | S822* P823* D824* H825* G826* K827* K828* |
| JPUL1037 | D77G A190I T197I N283F H321V D367N K370S S375H N401D S402Q Y412F |
| | F432V Q434E L435A T486V Q498R S531R A533I L581F Q595R D688A F700L |
| | V821* S822* P823* D824* H825* G826* K827* K828* |

### Example 3: Fermentation of the Bacillus Strains

B. subtilis strains were fermented on a rotary shaking table in 500 ml baffled flasks containing 100ml 10R-av-30C with 8 mg/L chloramphenicol at 200 rpm, 37° C. The culture broth was centrifuged (10,000×g, 20 min) and the supernatant was carefully decanted from the precipitates.

### Example 4: Purification of Pullulanases

Pullulanase variant was purified through two steps of ammonium sulfate precipitation and cation exchange chromatography. Finally, the sample was desalted and buffer exchanged using a centrifugal filter unit (Vivaspin Turbo 15, Sartorius) with 20 mM sodium acetate buffer pH 4.5. Enzyme concentrations were determined by A280 value.

### Example 5: Thermo-Stability Determination (TSA)

Purified enzyme was diluted with 50 mM sodium acetate buffer pH 4.5 to 0.5 mg/ml and mixed with equal volume of SYPRO Orange (Invitrogen) diluted with Milli-Q water. Eighteen ul of mixture solution were transfer to LightCycler 480 Multiwell Plate 384 (Roche Diagnostics) and the plate was sealed.

### Equipment Parameters of TSA

Apparatus: LightCycler 480 Real-Time PCR System (Roche Applied Science)
Scan rate: 0.02° C./sec
Scan range: 37-96° C.
Integration time: 1.0 sec
Excitation wave length 465 nm
Emission wave length 580 nm

The obtained fluorescence signal was normalized into a range of 0 and 1. The Td2 was defined as the temperature at the maximum signal intensity.

The thermo-stability improvements are listed in TABLE 3 with Td2 of JPUL604 as 0.

### Example 6: Pullulanase Assay

### PAHBAH-Maltodextrin (DE3) Assay

### Substrate Solution

1 g maltodextrin (pindex 100 from MATSUTANI chemical industry Co., Ltd.)
5 ml 50 mM sodium acetate buffer, pH 5

### PAHBAH Solution

0.0552 g Bismuth (III)-acetate
0.2 g PAHBAH
0.5 g Potassium sodium tartrate, tetrahydrate
10 ml 500 mM NaOH

Ten μl of enzyme samples were mixed with 110 μl of substrate solution and incubated at set temperatures for 2 hours (85° C.) or 30 min (91° C.). Ten ul of 0.5 N NaOH was added to stop reaction and cooled the tubes to 55° C. The reaction mixtures were diluted 40-fold with 50 mM sodium acetate buffer, pH 5. 40 μl of PAHBAH solution was added to 120 μl of diluted mixtures, incubated for another 20 min at 55° C. and the absorbance at A405 was read.

The activities are listed in TABLE 3 as relative activity to that of JPUL604.

TABLE 3

| | Increase in Td2 | Activity at 85° C. | Activity at 91° C. |
|---|---|---|---|
| | [° C.] | (Relative | (Relative |
| JPUL # | (JPUL604 as 0) | to JPUL604) | to JPUL604) |
| P609 | 0 | 100% | 127% |
| P624 | — | — | — |
| P625 | −0.16 | 97% | — |
| P629 | 0.12 | 118% | — |
| P630 | 0.54 | 137% | — |
| P631 | 0.69 | 129% | — |
| P632 | 0.96 | 140% | — |
| P633 | — | — | — |
| P634 | 0.25 | 130% | — |
| P635 | — | — | — |
| P638 | −0.69 | 104% | — |
| P639 | 1.56 | 93% | — |
| P640 | 0.32 | 127% | — |
| P641 | −0.42 | 108% | — |
| P643 | −0.06 | 120% | — |
| P644 | −0.51 | 109% | — |
| P645 | 0.19 | 126% | — |
| P646 | 0.45 | 136% | — |
| P648 | — | — | — |
| P649 | — | — | — |
| P650 | — | — | — |
| P651 | — | — | — |
| P652 | — | — | — |
| P653 | — | — | — |
| P654 | 1.16 | 119% | — |
| P655 | — | — | — |
| P656 | 2.68 | 99% | 105% |
| P657 | 1.60 | 122% | 92% |
| P675 | — | — | — |
| P676 | 2.98 | 132% | 44% |
| P677 | — | — | — |
| P678 | 3.41 | 132% | 56% |
| P679 | 3.50 | 133% | 103% |
| P680 | 3.53 | 139% | 111% |
| P681 | 3.29 | 119% | 63% |
| P682 | 3.38 | 134% | 43% |
| P683 | 3.96 | 149% | 84% |
| P684 | 3.56 | 157% | 123% |
| P685 | 4.18 | 129% | 147% |
| P698 | 4.42 | 130% | 136% |
| P699 | 4.07 | 126% | — |
| P700 | 4.07 | 135% | — |
| P703 | 4.24 | 133% | — |
| P704 | 3.63 | 119% | — |
| P718 | 2.68 | 183% | 225% |
| P719 | 2.87 | 185% | 238% |
| P725 | 4.00 | 135% | — |
| P730 | 2.80 | 189% | — |
| P731 | 3.01 | 181% | — |
| P738 | 2.62 | 173% | — |
| P740 | 1.99 | 151% | — |
| P741 | 3.24 | 227% | — |
| P743 | 2.49 | 180% | — |
| P745 | 2.80 | 179% | — |
| P746 | 3.21 | 187% | — |
| P747 | 2.58 | 182% | — |
| P753 | 3.39 | 204% | 217% |
| P755 | 2.94 | 194% | — |
| P762 | 3.19 | 187% | — |
| P763 | 3.09 | 204% | — |
| P764 | 3.15 | 211% | — |
| P765 | 3.52 | 216% | 242% |
| P766 | 2.97 | 229% | 226% |
| P767 | 3.32 | 205% | 207% |
| P771 | 3.00 | 196% | 210% |
| P776 | −0.06 | 88% | — |
| P777 | 3.37 | 197% | 224% |
| P778 | 4.04 | 225% | 223% |
| P779 | 3.70 | 237% | 233% |
| P780 | 3.88 | 223% | 243% |
| P781 | 3.74 | 215% | |
| P782 | 3.52 | 234% | 241% |
| P783 | 3.70 | 220% | 231% |
| P787 | 3.65 | 212% | 286% |
| P789 | 3.75 | 198% | 243% |
| P791 | 3.68 | 233% | 277% |
| P794 | 5.28 | 117% | 38% |
| P795 | 3.79 | 229% | 215% |
| P797 | 3.96 | 251% | 240% |
| P812 | 3.59 | 258% | 244% |
| P818 | 3.95 | 198% | 183% |
| P823 | 5.18 | 235% | 257% |
| P845 | 4.35 | 258% | 266% |
| P846 | 4.35 | 264% | 260% |
| P847 | 4.29 | 297% | 271% |
| P848 | 4.19 | 270% | 218% |
| P855 | 4.19 | 261% | 225% |
| P856 | 4.99 | 279% | 248% |
| P861 | 4.10 | 282% | 215% |
| P865 | 3.87 | — | 231% |
| P866 | 3.72 | — | 221% |
| P876 | 3.93 | — | 246% |
| P881 | 3.96 | — | 257% |
| P882 | 3.20 | — | 207% |
| P883 | 3.60 | — | 207% |
| P893 | 4.18 | — | 226% |
| P897 | 4.97 | — | 309% |
| P900 | — | — | — |
| P901 | 5.44 | — | 297% |
| P903 | 5.82 | — | 362% |
| P907 | 6.88 | — | 385% |
| P922 | 4.05 | — | 184% |
| P931 | 4.91 | — | 227% |
| P933 | 4.17 | — | 208% |
| P941 | 6.47 | — | 300% |
| P942 | — | — | 363% |
| P943 | — | — | 318% |
| P944 | 6.81 | — | 348% |
| P946 | 6.88 | — | 354% |
| P973 | — | — | — |
| P989 | — | — | — |
| P990 | 7.29 | — | 330% |
| P991 | 7.13 | — | 228% |
| P992 | 7.71 | — | 317% |
| P993 | 7.65 | — | 364% |
| P994 | 7.29 | — | 331% |
| P1025 | 3.83 | — | 236% |
| P1026 | 4.24 | — | 244% |
| P1031 | 7.03 | — | 348% |
| P1033 | 7.88 | — | 422% |
| P1034 | 7.00 | — | 335% |
| P1037 | 8.45 | — | 482% |

### Example 7. Application of Selected Variants in a Starch to Ethanol Process

Effect of JPUL604, JPUL609, JPUL719 variants enzyme addition in liquefaction process for increasing ethanol titer in simultaneous saccharification and fermentation process.

Liquefaction was carried out in a metal canister using Labomat BFA-24 (Mathis, Concord, NC). In the canister was added 37.2 g of industrial produced ground corn (88.7% dry solids) and 62.7 g tap water and mixed well. The target dry solid was about 33% DS. pH was adjusted to pH 5.0 and dry solid was measured using moisture balance (Mettler-Toledo). Alpha-Amylase BE369 was dosed 0.016% (w/w) into the corn slurry with or without appropriate amount of JPUL variant enzymes as shown in the table below. Total weight of corn slurry mixture was 100 g. As control, only Alpha-Amylase BE369 was added without addition of JPUL enzyme. Liquefaction took place in the Labomat chamber at 85° C. for 2 hr. After liquefaction, canister was cooled in ice-bath to room temperature and the respective liquefied mash was transferred to a container following by supplemented with 3 ppm of penicillin and 1000 ppm of urea. Simultaneous saccharification and fermentation (SSF) was performed via mini-scale fermentations using Ethanol Red™ yeast. Approximately 5 g of liquefied corn mash above was added to 15 ml tube vials. Each vial was dosed with 0.6 AGU/gDS of commercial glucoamylase Glucoamylase B blend followed by addition of 100 micro liters hydrated yeast per 5 g slurry. Actual glucoamylase dosage was based on the exact weight of liquefied corn mash in each vial. Vials were incubated at 32° C. Four replicates were used with 52 hours time point fermentation. Fermentation was stopped by addition of 50 micro liters of 40% H₂SO₄, follow by centrifuging, and filtering through a 0.2 micrometer filter. Ethanol and oligosaccharides concentration were determined using HPLC.

TABLE 4

| | Alpha-Amylase BE369 | JPUL dosage |
|---|---|---|
| Treatments | (%, w/w) | (μg/g DS) |
| 1. Control | 0.016 | — |
| 2. JPUL604 | 0.016 | 20 |
| 3. JPUL609 | 0.016 | 10 |
| 4. JPUL719 | 0.016 | 10 |

### Result

As shown in result table below, addition of JPUL variant enzymes together with Alpha-Amylase BE369 in liquefaction increased ethanol yield compared to control.

### Table

TABLE

| | Average ethanol |
|---|---|
| Treatments | concentration (g/l) |
| 1. Control | 133.32 |
| 2. JPUL604 | 133.34 |
| 3. JPUL609 | 133.49 |
| 4. JPUL719 | 133.50 |

1. A variant pullulanase, having increased thermo-stability and/or increased thermo-activity compared to a parent pullulanase, comprising a substitution at least at one position selected from a position corresponding to positions 432, 486, 370, 17, 77, 103, 106, 107, 190, 196, 197, 262, 279, 283, 321, 367, 375, 382, 399, 401, 402, 411, 412, 434, 435, 443, 446, 459, 460, 479, 490, 498, 514, 529, 531, 533, 541, 545, 581, 583, 595, 649, 665, 688, 700, 709, 804, and 811 of SEQ ID NO: 1, and optionally a deletion of one or more, e.g., all amino acids at positions 821, 822, 823, 824, 825, 826, 827, and 828, wherein the variant has pullulanase activity, and wherein the variant has at least 70%, at least 75%, at least 80%, at least 85%, at least 90%, at least 95%, at least 96%, at least 97%, at least 98%, or at least 99%, but less than 100% sequence identity to a parent alpha amylase selected from the group consisting of SEQ ID NO: 1, SEQ ID NO: 3, SEQ ID NO: 4, SEQ ID NO: 5.

2. The variant pullulanase of claim 1, comprising a substitution at a position corresponding to position 432 of SEQ ID NO: 1, wherein the variant pullulanase comprises valine in position 432 using SEQ ID NO: 1 for numbering, particularly a F432V substitution, wherein the variant has pullulanase activity, and wherein the variant has at least 70%, at least 75%, at least 80%, at least 85%, at least 90%, at least 95%, at least 96%, at least 97%, at least 98%, or at least 99%, but less than 100% sequence identity to a parent alpha amylase selected from the group consisting of SEQ ID NO: 1, SEQ ID NO: 3, SEQ ID NO: 4, SEQ ID NO: 5.

3. The variant pullulanase of claim 1, comprising a substitution at a position corresponding to position 486 of SEQ ID NO: 1, wherein the variant pullulanase comprises alanine or valine in position 486 using SEQ ID NO: 1 for numbering, particularly a T486A, V substitution, wherein the variant has pullulanase activity, and wherein the variant has at least 70%, at least 75%, at least 80%, at least 85%, at least 90%, at least 95%, at least 96%, at least 97%, at least 98%, or at least 99%, but less than 100% sequence identity to a parent alpha amylase selected from the group consisting of SEQ ID NO: 1, SEQ ID NO: 3, SEQ ID NO: 4, SEQ ID NO: 5.

4. The variant pullulanase of claim 1, comprising a substitution at a position corresponding to position 370 of SEQ ID NO: 1, wherein the variant pullulanase comprises serine in position 370 using SEQ ID NO: 1 for numbering, particularly a K370S substitution, wherein the variant has pullulanase activity, and wherein the variant has at least 70%, at least 75%, at least 80%, at least 85%, at least 90%, at least 95%, at least 96%, at least 97%, at least 98%, or at least 99%, but less than 100% sequence identity to a parent alpha amylase selected from the group consisting of SEQ ID NO: 1, SEQ ID NO: 3, SEQ ID NO: 4, SEQ ID NO: 5.

5. The variant pullulanase of any of the preceding claims 1, wherein the substitutions are selected from the group consisting of: K370S, F17Y, D77G, S103K, Q106W, A107D, A190I, V196T,C, T197I, T262V, Q279R, N283F, H321V, D367G,N, S375H, N382T, Q399N, N401D, S402Q, N411L, Y412F, F432V, Q434E, L435A, R443G, I446V, G459E, V460E, H479N, T486A,V, I490L, Q498R, V514A, T529L, S531R, A533I, N541D, A545I, L581F, N583D, Q595R, D649A, V665I, D688A, F700L, P709I, E804S, and G811R.

6. The variant pullulanase according to any of claims 1, wherein thermo-stability is determined as relative activity after heat stress for 30 min at two different temperatures, e.g., selected in the range from 60° C.-90° C., e.g., 70° C.-87° C., and subsequently assayed at 60-80° C., e.g., 70° C., using PHADEBAS assay.

7. The variant pullulanase according to any of claims 1, wherein thermo-stability is determined as increased melting (denaturing) temperature compared to the parent pullulanase using TSA assay.

8. The variant pullulanase according to any of claims 1, wherein thermo-activity is determined as relative activity determined at two different temperatures selected from the range of 70-86° C. using PHADEBAS assay.

9. The variant pullulanase according to any of the preceding claims 1, wherein the variant comprises combinations of substitutions and/or deletions selected from the group consisting of:
Q279R+K370S;
H321E+K370S;
K370S+V821*+S822*+P823*+D824*+H825*+G826*+K827*+K828*;
K370S+Q399N;
K370S+N401D;
K370S+F432V;
V196T+K370S;
V196C+K370S;
T197I+K370S;
K370S+V460E;
K370S+T486A;
K370S+T486V;
K370S+I490L;
K370S+V514A;
K370S+T529L;
K370S+S531R;
K370S+Q595R;
H321E+K370S+F432V;
K370S+F432V+V821*+S822*+P823*+D824*+H825*+G826*+K827*+K828*;
K370S+Q399N+F432V;
K370S+N401D+F432V;
V196T+K370S+F432V;
V196C+K370S+F432V;
T197I+K370S+F432V;
K370S+F432V+G459E;
K370S+F432V+T486A;
K370S+F432V+T486V;
V196T+K370S+F432V+T486A;
T197I+K370S+F432V+T486A;
V196T+T197I+K370S+F432V+T486A;
K370S+Q399N+F432V+T486A;
K370S+N401D+F432V+T486A;
K370S+Q399N+N401D+F432V+T486A;
K370S+S531R+F432V+T486A;
K370S+Q595R+F432V+T486A;
K370S+F432V+T486A+S531R+Q595R;
V196T+T197I+K370S+Q399N+N401D+F432V+T486A;
V196T+T197I+K370S+Q399N+N401D+F432V+T486A+Q595R;
T197I+K370S+N401D+F432V+T486A+S531R+Q595R;
T197I+K370S+N401D+F432V+T486A+S531R+Q595R+V821*+S822*+P823*+D824*+H825*+G826*+K827*+K828*;
T197I+K370S+Q399N+N401D+F432V+T486A+Q595R;
S103K+T197I+K370S+N401D+F432V+T486A+S531R+Q595R;
V196T+T197I+K370S+Q399N+N401D+F432V+T486V+Q595R;
T197I+K370S+N401D+F432V+T486V+S531R+Q595R;
V196T+T197I+K370S+Q399N+N401D+F432V+T486A+S531R+Q595R;
D77G+T197I+K370S+N401D+F432V+T486V+S531R+Q595R;
T197I+K370S+N401D+S402Q+F432V+T486V+S531R+Q595R;
T197I+N283F+K370S+N401D+F432V+T486V+S531R+Q595R;
T197I+N283F+K370S+N401D+S402Q+F432V+T486V+S531R+Q595R;
T197I+N283F+K370S+N401D+F432V+L435A+T486V+S531R+Q595R;
D77G+T197I+N283F+K370S+N401D+S402Q+F432V+T486V+S531R+Q595R;
T197I+N283F+K370S+N401D+S402Q+F432V+L435A+T486V+S531R+Q595R;
D77G+T197I+N283F+K370S+N401D+S402Q+F432V+L435A+T486V+S531R+Q595R;
T197I+N283F+K370S+N401D+S402Q+F432V+L435A+T486V+S531R+Q595R+D649A;
D77G+T197I+N283F+K370S+N401D+S402Q+F432V+L435A+T486V+S531R+Q595R+D649A;
D77G+Q106W+T197I+N283F+K370S+N401D+S402Q+F432V+L435A+T486V+S531R+Q595R;
D77G+A107D+T197I+N283F+K370S+N401D+S402Q+F432V+L435A+T486V+S531R+Q595R;
D77G+V196C+T197I+N283F+K370S+N401D+S402Q+F432V+L435A+T486V+S531R+Q595R;
D77G+T197I+N283F+K370S+N401D+S402Q+F432V+L435A+T486V+S531R+N583D+Q595R;
D77G+T197I+N283F+K370S+N401D+S402Q+F432V+L435A+T486V+S531R+Q595R+V821*+S822*+P823*+D824*+H825*+G826*+K827*+K828*;
D77G+T197I+N283F+K370S+N401D+S402Q+F432V+L435A+T486V+S531R+Q595R+D688A+V821*+S822*+P823*+D824*+H825*+G826*+K827*+K828*;
D77G+T197I+N283F+D367G+K370S+N401D+S402Q+F432V+L435A+T486V+S531R+Q595R+V821*+S822*+P823*+D824*+H825*+G826*+K827*+K828*;
D77G+T197I+N283F+D367N+K370S+N401D+S402Q+F432V+L435A+T486V+S531R+Q595R+V821*+S822*+P823*+D824*+H825*+G826*+K827*+K828*;
D77G+T197I+N283F+K370S+S375H+N401D+S402Q+F432V+L435A+T486V+S531R+Q595R+V821*+S822*+P823*+D824*+H825*+G826*+K827*+K828*;
D77G+T197I+N283F+K370S+N382T+N401D+S402Q+F432V+L435A+T486V+S531R+Q595R+V821*+S822*+P823*+D824*+H825*+G826*+K827*+K828*;
D77G+T197I+N283F+K370S+N401D+S402Q+N411L+F432V+L435A+T486V+S531R+Q595R+V821*+S822*+P823*+D824*+H825*+G826*+K827*+K828*;
D77G+T197I+N283F+K370S+N401D+S402Q+Y412F+F432V+L435A+T486V+S531R+Q595R+V821*+S822*+P823*+D824*+H825*+G826*+K827*+K828*;
D77G+T197I+N283F+K370S+N401D+S402Q+F432V+Q434E+L435A+T486V+S531R+Q595R+V821*+S822*+P823*+D824*+H825*+G826*+K827*+K828*;
D77G+T197I+N283F+K370S+N401D+S402Q+F432V+L435A+R443G+T486V+S531R+Q595R+V821*+S822*+P823*+D824*+H825*+G826*+K827*+K828*;
D77G+T197I+N283F+K370S+N401D+S402Q+F432V+L435A+I446V+T486V+S531R+Q595R+V821*+S822*+P823*+D824*+H825*+G826*+K827*+K828*;
D77G+T197I+N283F+K370S+N401D+S402Q+F432V+L435A+T486V+Q498R+S531R+Q595R+V821*+S822*+P823*+D824*+H825*+G826*+K827*+K828*;
D77G+T197I+N283F+K370S+N401D+S402Q+F432V+L435A+T486V+S531R+A533I+Q595R+V821*+S822*+P823*+D824*+H825*+G826*+K827*+K828*;
D77G+T197I+N283F+K370S+N401D+S402Q+F432V+L435A+T486V+S531R+N541D+Q595R+V821*+S822*+P823*+D824*+H825*+G826*+K827*+K828*;
D77G+T197I+N283F+K370S+N401D+S402Q+F432V+L435A+T486V+S531R+A545I+Q595R+V821*+S822*+P823*+D824*+H825*+G826*+K827*+K828*;
D77G+T197I+N283F+K370S+N401D+S402Q+F432V+L435A+T486V+S531R+L581F+Q595R+V821*+S822*+P823*+D824*+H825*+G826*+K827*+K828*;
D77G+T197I+N283F+K370S+N401D+S402Q+F432V+L435A+H479N+T486V+S531R+Q595R+D688A+V821*+S822*+P823*+D824*+H825*+G826*+K827*+K828*;
D77G+T197I+N283F+D367N+K370S+N401D+S402Q+F432V+L435A+T486V+S531R+Q595R+D688A+V821*+S822*+P823*+D824*+H825*+G826*+K827*+K828*;
D77G+T197I+N283F+K370S+S375H+N401D+S402Q+F432V+L435A+T486V+S531R+Q595R+D688A+V821*+S822*+P823*+D824*+H825*+G826*+K827*+K828*;
D77G+T197I+N283F+K370S+N401D+S402Q+Y412F+F432V+L435A+T486V+S531R+Q595R+D688A+V821*+S822*+P823*+D824*+H825*+G826*+K827*+K828*;
D77G+T197I+N283F+K370S+N401D+S402Q+F432V+L435A+T486V+S531R+A533I+Q595R+D688A+V821*+S822*+P823*+D824*+H825*+G826*+K827*+K828*;
D77G+T197I+N283F+K370S+N401D+S402Q+F432V+L435A+T486V+S531R+L581F+Q595R+D688A+V821*+S822*+P823*+D824*+H825*+G826*+K827*+K828*;
D77G T197I N283F D367N K370S S375H N401D S402Q F432V L435A T486V S531R Q595R D688A V821*S822*P823*D824*H825*G826*K827*K828*;
D77G T197I N283F D367N K370S N401D S402Q Y412F F432V L435A T486V S531R Q595R D688A V821*S822*P823*D824*H825*G826*K827*K828*;
D77G T197I N283F D367N K370S N401D S402Q F432V L435A T486V S531R A533| Q595R D688A V821*S822*P823*D824*H825*G826*K827*K828*;
D77G T197I N283F D367N K370S N401D S402Q F432V L435A T486V S531R L581F Q595R D688A V821*S822*P823*D824*H825*G826*K827*K828*;
D77G T197I N283F K370S N401D S402Q F432V L435A T486V S531R Q595R V665I V821*S822*P823*D824*H825*G826*K827*K828*;
D77G T197I N283F K370S N401D S402Q F432V L435A T486V S531R Q595R F700L V821*S822*P823*D824*H825*G826*K827*K828*;
D77G T197I N283F K370S N401D S402Q F432V L435A T486V S531R Q595R P709I V821*S822*P823*D824*H825*G826*K827*K828*;
D77G T197I N283F D367N K370S S375H N401D S402Q Y412F F432V L435A T486V S531R Q595R D688A V821*S822*P823*D824*H825*G826*K827*K828*;
D77G T197I N283F D367N K370S S375H N401D S402Q F432V L435A T486V S531R A533I Q595R D688A V821*S822*P823*D824*H825*G826*K827*K828*;
D77G T197I N283F D367N K370S S375H N401D S402Q Y412F F432V L435A T486V S531R A533I L581F Q595R D688A V821*S822*P823*D824*H825*G826*K827*K828*;
D77G T197I N283F H321V D367N K370S S375H N401D S402Q Y412F F432V Q434E L435A T486V S531R A533I L581F Q595R D688A V821*S822*P823*D824*H825*G826*K827*K828*;
D77G T197I N283F H321V D367N K370S S375H N401D S402Q Y412F F432V L435A T486V Q498R S531R A533I L581F Q595R D688A V821*S822*P823*D824*H825*G826*K827*K828*;
D77G T197I N283F H321V D367N K370S S375H N401D S402Q Y412F F432V L435A T486V S531R A533I L581F Q595R D688A F700L V821*S822*P823*D824*H825*G826*K827*K828*;
D77G T197I N283F H321V D367N K370S S375H N401D S402Q Y412F F432V Q434E L435A T486V Q498R S531R A533I L581F Q595R D688A F700L V821*S822*P823*D824*H825*G826*K827*K828*;
F17Y D77G T197I N283F K370S N401D S402Q F432V L435A T486V S531R Q595R V821*S822*P823*D824*H825*G826*K827*K828*;
D77G T197I N283F K370S N401D S402Q F432V L435A T486V S531R Q595R E804S V821*S822*P823*D824*H825*G826*K827*K828*;
D77G T197I N283F K370S N401D S402Q F432V L435A T486V S531R Q595R G811R V821*S822*P823*D824*H825*G826*K827*K828*;
D77G T197I N283F H321V K370S S375H N401D S402Q Y412F F432V Q434E L435A H479N T486V Q498R S531R A533I L581F Q595R D688A F700L V821*S822*P823*D824*H825*G826*K827*K828*;
D77G A190I T197I N283F K370S S375H N401D S402Q Y412F F432V Q434E L435A H479N T486V Q498R S531R A533I L581F Q595R D688A F700L V821*S822*P823*D824*H825*G826*K827*K828*;
D77G T197I T262V N283F K370S S375H N401D S402Q Y412F F432V Q434E L435A H479N T486V Q498R S531R A533I L581F Q595R D688A F700L V821*S822*P823*D824*H825*G826*K827*K828*; and
D77G A190I T197I N283F H321V D367N K370S S375H N401D S402Q Y412F F432V Q434E L435A T486V Q498R S531R A533I L581F Q595R D688A F700L V821*S822*P823*D824*H825*G826*K827*K828*.

10. The variant pullulanase of any of the preceding claims 1, comprising the substitutions N222P+Q252A+Q256R+N368G+N393A+Q431E+N610R+G624S+T631S+S632C, and optionally L432F.

11. The variant pullulanase of any of the preceding claims 1, comprising the substitutions N222P+Q252A+Q256R+N368G+N393A+Q431E+N610R+G624S+T631S+S632C+N20G+Y28K+H80Y+Q187R+E310A+D311K+Q387L+Q459G+D586S+E699R+S798R, and optionally L432F.

12. The variant pullulanase of any of the preceding claims 1, comprising the deletions P30*+V31*+N32*, and optionally Q29G.

13. The variant pullulanase of any of the preceding claims 1, comprising the modifications Q29G+P30*+V31*+N32*+D57N+D58P+A195G+N202K+A345P+F456W+E560R, and optionally one, two, three, four or five of N197T, M402S, I460V, N479H, I514V.

14. The variant pullulanase of any of the preceding claims 1, comprising the modifications N222P+Q252A+Q256R+N368G+N393A+Q431E++N610R+G624S+T631S+S632C+N20G+Y28K+H80Y+Q187R+E310A+D311K+Q387L+Q459G+D586S+E699R+S798R+Q29G+P30*+V31*+N32*+D57N+D58P+A195G+N202K+A345P+F456W+E560R, and optionally one, two, three, four, five or six of L432F, N197T, M402S, N479H, I460V, I514V.

15. The variant of any of the preceding claims 1, wherein the variant comprises the substitutions X370S+X432V, particularly K370S+L,F432V, and optionally X492A,S.

16. The variant according to any of claims 1, wherein the variants have has an increase in thermo-activity relative to a parent pullulanase, e.g., the pullulanase disclosed as SEQ ID NO: 3, of at least 5%, at least 10%, at least 20%, at least 25%, at least 30%, at least 35%, at least 40%, at least 50%, at least 60%, at least 70%, at least 80%, at least 90%, at least 100%, after enzymatic reaction of maltodextrin 2 hours at 85° C. or 30 min 91° C., and subsequent determination of digested maltodextrin fraction by PAHBAH assay at 55° C.

17. The variants of any of the preceding claims 1, wherein the increase in thermo-stability determined as increased melting (denaturing) temperature compared to the parent pullulanase disclosed in SEQ ID NO: 3 using TSA assay is at least 0.3 degrees C., at least 0.4 degrees C., at least 0.5 degrees C., at least 0.6 degrees C., at least 0.8 degrees C., at least 1.0 degrees C., at least 1.2 degrees C., at least 1.5 degrees C., at least 2.0 degrees C., at least 2.5 degrees C., at least 3.0 degrees C., at least 3.5 degrees C., at least 4.0 degrees C., at least 4.5 degrees C., at least 5.0 degrees C.

18. A polynucleotide encoding the variant pullulanase of any of the claims 1.

19. A nucleic acid construct or expression vector comprising the polynucleotide of claim 18 operably linked to one or more control sequences that direct the production of the polypeptide in an expression host.

20. A recombinant host cell comprising the polynucleotide of claim 18 operably linked to one or more control sequences that direct the production of the polypeptide.

21. A composition comprising the variant pullulanase of any of claims 1 and a stabilizer.

22. A method of producing a variant pullulanase according to any of the claims 1, comprising cultivating the host cell of claim 20 under conditions conducive for production of the polypeptide.

23. A process for producing a syrup from starch-containing material comprising the steps of:
a) liquefying the starch-containing material at a temperature above the initial gelatinization temperature using an alpha-amylase and a variant pullulanase of any of the claims 1;
b) saccharifying using a glucoamylase.

24. A process for producing fermentation products from starch-containing material comprising the steps of:
a) liquefying the starch-containing material at a temperature above the initial gelatinization temperature using an alpha-amylase and a variant pullulanase of any of the claims 1;
b) saccharifying using a glucoamylase;
c) fermenting using a fermenting organism.

25. (canceled)

26. (canceled)

27. (canceled)

28. A method of producing a brewer's wort comprising adding to a mash, a pullulanase of any of the claims 1.
